SEQUENCE LISTING (223 sequences)

SEQ ID NO: 1 - DNA, 19 bp, Artificial Sequence
  Primer
gtctgaggcctcccttttt

SEQ ID NO: 2 - DNA, 20 bp, Artificial Sequence
  Primer
gggagaccaaagccttcata

SEQ ID NO: 3 - DNA, 21 bp, Artificial Sequence
  Primer
tggagaacattgacgtgtgtg

SEQ ID NO: 4 - DNA, 21 bp, Artificial Sequence
  Primer
atccctcataggtgaccagtg

SEQ ID NO: 5 - DNA, 20 bp, Artificial Sequence
  Primer
ccacggagctcctgttttac

SEQ ID NO: 6 - DNA, 20 bp, Artificial Sequence
  Primer
cagcttcctcttcagcaggt

SEQ ID NO: 7 - DNA, 21 bp, Artificial Sequence
  Primer
tgctcatctttcatcgacgga

SEQ ID NO: 8 - DNA, 22 bp, Artificial Sequence
  Primer
agtggctttgggtcacatattc

SEQ ID NO: 9 - DNA, 21 bp, Artificial Sequence
  Primer
agtctctgcacatcgttatgc

SEQ ID NO: 10 - DNA, 21 bp, Artificial Sequence
  Primer
ggtgttgaagccgatagtggg

SEQ ID NO: 11 - DNA, 20 bp, Artificial Sequence
  Primer
cctgggaaacccgtgtactc

SEQ ID NO: 12 - DNA, 23 bp, Artificial Sequence
  Primer
agatccaccattttgcactcatt

SEQ ID NO: 13 - DNA, 21 bp, Artificial Sequence
  Primer
agctgaaatacgctgacatcc

SEQ ID NO: 14 - DNA, 20 bp, Artificial Sequence
  Primer
ctcactcttcacgggttggg

SEQ ID NO: 15 - DNA, 22 bp, Artificial Sequence
  Primer
catgatagaccagtccaaccga

SEQ ID NO: 16 - DNA, 21 bp, Artificial Sequence
  Primer
attggctgtccatcagggtta

SEQ ID NO: 17 - DNA, 21 bp, Artificial Sequence
  Primer
tgacggatacagaggtgtcat

SEQ ID NO: 18 - DNA, 21 bp, Artificial Sequence
  Primer
acgctacacggatgtacttga

SEQ ID NO: 19 - DNA, 23 bp, Artificial Sequence
  Primer
gttaatgggaacttcagtgacca

SEQ ID NO: 20 - DNA, 21 bp, Artificial Sequence
  Primer
ctgcgtgtaattcagaaggct

SEQ ID NO: 21 - DNA, 22 bp, Artificial Sequence
  Primer
ttccctacagaactcttgagcc

SEQ ID NO: 22 - DNA, 19 bp, Artificial Sequence
  Primer
ggggcactctccttcctga

SEQ ID NO: 23 - DNA, 29 bp, Artificial Sequence
  Primer
aggtttttgttgtttttgtagtttttatt

SEQ ID NO: 24 - DNA, 26 bp, Artificial Sequence
  Primer
accaccctaaaaacctaatttctatc

SEQ ID NO: 25 - DNA, 23 bp, Artificial Sequence
  Primer
ttttttggggaggatagaaagag

SEQ ID NO: 26 - DNA, 25 bp, Artificial Sequence
  Primer
tcccaccctcctaaatataaaactt

SEQ ID NO: 27 - DNA, 27 bp, Artificial Sequence
  Primer
tttttatttgtatttttgtatgtttta

SEQ ID NO: 28 - DNA, 28 bp, Artificial Sequence
  Primer
aaagggttaattgaaaggtttagtttaa

SEQ ID NO: 29 - DNA, 25 bp, Artificial Sequence
  Primer
aaaacaaaaataaccaacaaccaac

SEQ ID NO: 30 - DNA, 26 bp, Artificial Sequence
  Primer
ggagtaggagtattaggaaatataaa

SEQ ID NO: 31 - DNA, 25 bp, Artificial Sequence
  Primer
taacaaaatcaaaaaataaaaaaaa

SEQ ID NO: 32 - DNA, 25 bp, Artificial Sequence
  Primer
ggagtaggagtattaggaaatataa

SEQ ID NO: 33 - DNA, 25 bp, Artificial Sequence
  Primer
aattttatatttaagttttgtgttt

SEQ ID NO: 34 - DNA, 25 bp, Artificial Sequence
  Primer
aggatattttagttatagtttaggg

SEQ ID NO: 35 - DNA, 30 bp, Artificial Sequence
  Primer
gttatgtgtaatagaaatagtttattaaga

SEQ ID NO: 36 - DNA, 25 bp, Artificial Sequence
  Primer
aactaaaaataaaacacaatacctc

SEQ ID NO: 37 - DNA, 26 bp, Artificial Sequence
  Primer
gaagttagtttgtggggtttttaatt

SEQ ID NO: 38 - DNA, 25 bp, Artificial Sequence
  Primer
actaacaaaaataactttccattcc

SEQ ID NO: 39 - DNA, 25 bp, Artificial Sequence
  Primer
tatagtatttggagtaggttttagt

SEQ ID NO: 40 - DNA, 25 bp, Artificial Sequence
  Primer
agtatttggagtaggttttagtatg

SEQ ID NO: 41 - DNA, 26 bp, Artificial Sequence
  Primer
ccaaaactaatatccttaatttaatc

SEQ ID NO: 42 - DNA, 24 bp, Artificial Sequence
  Primer
gtaggaagggttgtttttgttttt

SEQ ID NO: 43 - DNA, 30 bp, Artificial Sequence
  Primer
ccaaacccacataataaaatatattaccta

SEQ ID NO: 44 - DNA, 24 bp, Artificial Sequence
  Primer
gtaggaagggttgtttttgttttt

SEQ ID NO: 45 - DNA, 25 bp, Artificial Sequence
  Primer
taggttattattgtgaaggttagag

SEQ ID NO: 46 - DNA, 25 bp, Artificial Sequence
  Primer
ttttaggaaattggttggtataaaa

SEQ ID NO: 47 - DNA, 25 bp, Artificial Sequence
  Primer
tataaatcctcaaaaaccaaacata

SEQ ID NO: 48 - DNA, 25 bp, Artificial Sequence
  Primer
agtttttgattttaggtttggtgag

SEQ ID NO: 49 - DNA, 25 bp, Artificial Sequence
  Primer
aaaaataacttctccacctatcaac

SEQ ID NO: 50 - DNA, 25 bp, Artificial Sequence
  Primer
agtttttgattttaggtttggtgag

SEQ ID NO: 51 - DNA, 25 bp, Artificial Sequence
  Primer
gggtaagggagataaaataaaataa

SEQ ID NO: 52 - DNA, 25 bp, Artificial Sequence
  Primer
agttttttagggtatttagttttag

SEQ ID NO: 53 - DNA, 30 bp, Artificial Sequence
  Primer
tatttatgggtaaagaagaaatgtagtata

SEQ ID NO: 54 - DNA, 27 bp, Artificial Sequence
  Primer
aaatctaaaaatttaaacaataacctc

SEQ ID NO: 55 - DNA, 25 bp, Artificial Sequence
  Primer
atgaagttgggataagatagttttg

SEQ ID NO: 56 - DNA, 25 bp, Artificial Sequence
  Primer
tctacaaccaactctaccaacaaac

SEQ ID NO: 57 - DNA, 25 bp, Artificial Sequence
  Primer
atgaagttgggataagatagttttg

SEQ ID NO: 58 - DNA, 25 bp, Artificial Sequence
  Primer
agtagtttggtgtttttttatatgt

SEQ ID NO: 59 - DNA, 24 bp, Artificial Sequence
  Primer
aggaggtggtggtgatttagtaag

SEQ ID NO: 60 - DNA, 26 bp, Artificial Sequence
  Primer
cccctatttaaaaatttaactctcac

SEQ ID NO: 61 - DNA, 25 bp, Artificial Sequence
  Primer
ttttgttatatttttattgtttgat

SEQ ID NO: 62 - DNA, 27 bp, Artificial Sequence
  Primer
aaaaaataaactacacttatcaatatc

SEQ ID NO: 63 - DNA, 25 bp, Artificial Sequence
  Primer
ttttgttatatttttattgtttgat

SEQ ID NO: 64 - DNA, 24 bp, Artificial Sequence
  Primer
gtatttttttttttaaatagttaa

SEQ ID NO: 65 - DNA, 24 bp, Artificial Sequence
  Primer
aatggaaatataattaggagagta

SEQ ID NO: 66 - DNA, 30 bp, Artificial Sequence
  Primer
ggagttgtttattttttttaaagtttaagt

SEQ ID NO: 67 - DNA, 25 bp, Artificial Sequence
  Primer
aactaaaatcaaatcctatcttttt

SEQ ID NO: 68 - DNA, 25 bp, Artificial Sequence
  Primer
tatgggagggtttataattgtgtag

SEQ ID NO: 69 - DNA, 25 bp, Artificial Sequence
  Primer
tataaaaaatctctcccaccaacat

SEQ ID NO: 70 - DNA, 25 bp, Artificial Sequence
  Primer
tatgggagggtttataattgtgtag

SEQ ID NO: 71 - DNA, 25 bp, Artificial Sequence
  Primer
gtggagagaagatgaagatagtatg

SEQ ID NO: 72 - DNA, 25 bp, Artificial Sequence
  Primer
taggttgttggatgaagttattttg

SEQ ID NO: 73 - DNA, 25 bp, Artificial Sequence
  Primer
tttttggatatataattttttaatt

SEQ ID NO: 74 - DNA, 24 bp, Artificial Sequence
  Primer
taaaaactcaaaaaaaacccaatc

SEQ ID NO: 75 - DNA, 28 bp, Artificial Sequence
  Primer
tttatgttatttttttattaattttatg

SEQ ID NO: 76 - DNA, 30 bp, Artificial Sequence
  Primer
taacttccaaaacaaaactctaaaatatct

SEQ ID NO: 77 - DNA, 25 bp, Artificial Sequence
  Primer
ttatttttttattaattttatgttt

SEQ ID NO: 78 - DNA, 25 bp, Artificial Sequence
  Primer
ttttattaattttatttttttttta

SEQ ID NO: 79 - DNA, 25 bp, Artificial Sequence
  Primer
attatgggatagtggtagtttttat

SEQ ID NO: 80 - DNA, 25 bp, Artificial Sequence
  Primer
ggtttgaaggagttgtttttaagtt

SEQ ID NO: 81 - DNA, 27 bp, Artificial Sequence
  Primer
tttgtttttgaatttagagaaaggaaa

SEQ ID NO: 82 - DNA, 28 bp, Artificial Sequence
  Primer
aaccttaaaaccaaatccattaataatc

SEQ ID NO: 83 - DNA, 30 bp, Artificial Sequence
  Primer
tttatggttttttataagaaatgatttata

SEQ ID NO: 84 - DNA, 25 bp, Artificial Sequence
  Primer
ctctaaactccaaaaactacactcc

SEQ ID NO: 85 - DNA, 30 bp, Artificial Sequence; identical to SEQ ID NO: 83
  Primer

SEQ ID NO: 86 - DNA, 25 bp, Artificial Sequence
  Primer
agttattggatttgtttagtgtagt

SEQ ID NO: 87 - DNA, 25 bp, Artificial Sequence
  Primer
taggattttgaattattttgattat

SEQ ID NO: 88 - DNA, 22 bp, Artificial Sequence
  Primer
ctcctaaaccccaactaaatct

SEQ ID NO: 89 - DNA, 25 bp, Artificial Sequence
  Primer
gtttggggatatttagataggatgt

SEQ ID NO: 90 - DNA, 25 bp, Artificial Sequence
  Primer
ttaaacacccacaaaattaattttt

SEQ ID NO: 91 - DNA, 25 bp, Artificial Sequence
  Primer
tttagataggatgtggaaagtttgt

SEQ ID NO: 92 - DNA, 25 bp, Artificial Sequence
  Primer
attagggttattttgttaaagaaaa

SEQ ID NO: 93 - DNA, 27 bp, Artificial Sequence
  Primer
ggagtggttggattataaatattgtgt

SEQ ID NO: 94 - DNA, 25 bp, Artificial Sequence
  Primer
taatttccccatcaccttaaaatta

SEQ ID NO: 95 - DNA, 25 bp, Artificial Sequence
  Primer
gatagtttaggttgggagggttaat

SEQ ID NO: 96 - DNA, 25 bp, Artificial Sequence
  Primer
tcctatcccaatattttctccatac

SEQ ID NO: 97 - DNA, 25 bp, Artificial Sequence
  Primer
gatagtttaggttgggagggttaat

SEQ ID NO: 98 - DNA, 25 bp, Artificial Sequence
  Primer
gtttttttttttttttttttatttt

SEQ ID NO: 99 - DNA, 25 bp, Artificial Sequence
  Primer
gttttagatatttgtagttttgaat

SEQ ID NO: 100 - DNA, 25 bp, Artificial Sequence
  Primer
ttgtgtattggtaaagtggtgggga

SEQ ID NO: 101 - DNA, 25 bp, Artificial Sequence
  Primer
attttaaggtgatggggaaattagt

SEQ ID NO: 102 - DNA, 25 bp, Artificial Sequence
  Primer
ataacaaaaattccaaaacttacac

SEQ ID NO: 103 - DNA, 25 bp, Artificial Sequence
  Primer
tggattttaggttttaggtttgaga

SEQ ID NO: 104 - DNA, 25 bp, Artificial Sequence
  Primer
aacactactcaaaccacccaataac

SEQ ID NO: 105 - DNA, 25 bp, Artificial Sequence
  Primer
agaattttagttttaggttagttta

SEQ ID NO: 106 - DNA, 25 bp, Artificial Sequence
  Primer
gtttattttataggtagagttagaa

SEQ ID NO: 107 - DNA, 30 bp, Artificial Sequence
  Primer
tttaggagtgagtatatttaatataatagg

SEQ ID NO: 108 - DNA, 25 bp, Artificial Sequence
  Primer
ttcataactaaaaacacaaaaaaaa

SEQ ID NO: 109 - DNA, 27 bp, Artificial Sequence
  Primer
gattttttattagtaaattaaaggtag

SEQ ID NO: 110 - DNA, 25 bp, Artificial Sequence
  Primer
aatttccctcaatcatcacaaatac

SEQ ID NO: 111 - DNA, 25 bp, Artificial Sequence
  Primer
gattttttattagtaaattaaaggt

SEQ ID NO: 112 - DNA, 23 bp, Artificial Sequence
  Primer
gttgtattataatagttttgtgg

SEQ ID NO: 113 - DNA, 25 bp, Artificial Sequence
  Primer
tgtttattgggttttttgggtttta

SEQ ID NO: 114 - DNA, 26 bp, Artificial Sequence
  Primer
gaaattttgagagtttttttgtagtt

SEQ ID NO: 115 - DNA, 27 bp, Artificial Sequence
  Primer
acatcaaaaccctaccattatcttaaa

SEQ ID NO: 116 - DNA, 25 bp, Artificial Sequence
  Primer
ttgtagggtgtaagttataaatgtt

SEQ ID NO: 117 - DNA, 25 bp, Artificial Sequence
  Primer
ttaataaccaacaaccccaactaat

SEQ ID NO: 118 - DNA, 24 bp, Artificial Sequence
  Primer
ttgtagggtgtaagttataaatgt

SEQ ID NO: 119 - DNA, 25 bp, Artificial Sequence
  Primer
gtgttgtagattttatttaatgatt

SEQ ID NO: 120 - DNA, 24 bp, Artificial Sequence
  Primer
gaggattttttagttagaaagttg

SEQ ID NO: 121 - DNA, 26 bp, Artificial Sequence
  Primer
actctataccactctaattcaatccc

SEQ ID NO: 122 - DNA, 25 bp, Artificial Sequence
  Primer
ttttattaattagttggggttgttg

SEQ ID NO: 123 - DNA, 25 bp, Artificial Sequence
  Primer
taacctaccaaaaaacaaaaaatac

SEQ ID NO: 124 - DNA, 25 bp, Artificial Sequence
  Primer
ttttattaattagttggggttgttg

SEQ ID NO: 125 - DNA, 25 bp, Artificial Sequence
  Primer
agggttttgatgttttttgatagtt

SEQ ID NO: 126 - DNA, 25 bp, Artificial Sequence
  Primer
tttagttttttttattttatttggg

SEQ ID NO: 127 - DNA, 25 bp, Artificial Sequence
  Primer
aaaaatctttattccctttcatctc

SEQ ID NO: 128 - DNA, 26 bp, Artificial Sequence
  Primer
ggtttgtggttatttttgaattagtt

SEQ ID NO: 129 - DNA, 28 bp, Artificial Sequence
  Primer
actcccccattttaatataattattacc

SEQ ID NO: 130 - DNA, 26 bp, Artificial Sequence
  Primer
ggtttgtggttatttttgaattagtt

SEQ ID NO: 131 - DNA, 25 bp, Artificial Sequence
  Primer
gattagttgtattaggagattgtat

SEQ ID NO: 132 - DNA, 25 bp, Artificial Sequence
  Primer
atgattatatagagaggtttggtag

SEQ ID NO: 133 - DNA, 26 bp, Artificial Sequence
  Primer
aggttttggagaagagtagttttaag

SEQ ID NO: 134 - DNA, 30 bp, Artificial Sequence
  Primer
aaaattcaaatcttcataaaatacaaatac

SEQ ID NO: 135 - DNA, 25 bp, Artificial Sequence
  Primer
aggttttgtatagagatgagtttag

SEQ ID NO: 136 - DNA, 25 bp, Artificial Sequence
  Primer
aaaaaacaaaccaaaccctaataac

SEQ ID NO: 137 - DNA, 25 bp, Artificial Sequence
  Primer
taggttttgtatagagatgagttta

SEQ ID NO: 138 - DNA, 25 bp, Artificial Sequence
  Primer
atgttttataggagtgatttatatt

SEQ ID NO: 139 - DNA, 25 bp, Artificial Sequence
  Primer
gtgttgtgagggaagtgggttgatg

SEQ ID NO: 140 - DNA, 26 bp, Artificial Sequence
  Primer
ttgtgtattgtttatgattttataat

SEQ ID NO: 141 - DNA, 30 bp, Artificial Sequence
  Primer
acaattcctttctatatttatttaatattt

SEQ ID NO: 142 - DNA, 25 bp, Artificial Sequence
  Primer
taatgttttgggtgtttttggtagt

SEQ ID NO: 143 - DNA, 30 bp, Artificial Sequence
  Primer
ttcctatcttaacctcttatttcaactata

SEQ ID NO: 144 - DNA, 25 bp, Artificial Sequence
  Primer
tgttttgggtgtttttggtagtttt

SEQ ID NO: 145 - DNA, 25 bp, Artificial Sequence
  Primer
agaattttagttttaggttagttta

SEQ ID NO: 146 - DNA, 25 bp, Artificial Sequence
  Primer
gtttattttataggtagagttagaa

SEQ ID NO: 147 - DNA, 25 bp, Artificial Sequence
  Primer
ttttgtaaagttatttttgagtttt

SEQ ID NO: 148 - DNA, 26 bp, Artificial Sequence
  Primer
ttaataacaaaaactaattattttcc

SEQ ID NO: 149 - DNA, 27 bp, Artificial Sequence
  Primer
ggggagtgattgtttgagtaattataa

SEQ ID NO: 150 - DNA, 24 bp, Artificial Sequence
  Primer
acccacattcatcacaataaaaaa

SEQ ID NO: 151 - DNA, 25 bp, Artificial Sequence
  Primer
tgtaatttggtttagttatttttat

SEQ ID NO: 152 - DNA, 25 bp, Artificial Sequence
  Primer
aattaggggtatttattgatttatg

SEQ ID NO: 153 - DNA, 30 bp, Artificial Sequence
  Primer
taatttaaattggtttttttgtgtttatta

SEQ ID NO: 154 - DNA, 27 bp, Artificial Sequence
  Primer
caatacctatattccaaatcaatatcc

SEQ ID NO: 155 - DNA, 25 bp, Artificial Sequence
  Primer
tattttaaattttgtttagggaggt

SEQ ID NO: 156 - DNA, 30 bp, Artificial Sequence
  Primer
tacaataaaatcaaaccctataatatatca

SEQ ID NO: 157 - DNA, 25 bp, Artificial Sequence
  Primer
tattttaaattttgtttagggaggt

SEQ ID NO: 158 - DNA, 25 bp, Artificial Sequence
  Primer
atagatgaaatgggtattttattag

SEQ ID NO: 159 - DNA, 30 bp, Artificial Sequence
  Primer
ggaatataggtattgtgagtataggaaagt

SEQ ID NO: 160 - DNA, 27 bp, Artificial Sequence
  Primer
tcaatttaaaaaccataaacaaaaatc

SEQ ID NO: 161 - DNA, 30 bp, Artificial Sequence
  Primer
atttggtatatgtaatgttataaaataaat

SEQ ID NO: 162 - DNA, 25 bp, Artificial Sequence
  Primer
aatcataacttcctacttaaaaccc

SEQ ID NO: 163 - DNA, 30 bp, Artificial Sequence; identical to SEQ ID NO: 161
  Primer

SEQ ID NO: 164 - DNA, 25 bp, Artificial Sequence
  Primer
tagaatttttagattatgggattag

SEQ ID NO: 165 - DNA, 25 bp, Artificial Sequence
  Primer
ttaaatatataagaaaggagtatgg

SEQ ID NO: 166 - DNA, 30 bp, Artificial Sequence
  Primer
ttttataattttattatttattttggtttt

SEQ ID NO: 167 - DNA, 28 bp, Artificial Sequence
  Primer
aaaaaatttacacacattcaatacaaac

SEQ ID NO: 168 - DNA, 25 bp, Artificial Sequence
  Primer
gagagagtttgttaattttgttttt

SEQ ID NO: 169 - DNA, 30 bp, Artificial Sequence
  Primer
accctaaaatatatcacctataaacctata

SEQ ID NO: 170 - DNA, 25 bp, Artificial Sequence
  Primer
gagagagtttgttaattttgttttt

SEQ ID NO: 171 - DNA, 25 bp, Artificial Sequence
  Primer
tttattggtttaaagatatttttag

SEQ ID NO: 172 - DNA, 30 bp, Artificial Sequence
  Primer
gattagagatgtaaattgtagtgtagagtt

SEQ ID NO: 173 - DNA, 25 bp, Artificial Sequence
  Primer
acaaaaaaaaccaacaaccaataaa

SEQ ID NO: 174 - DNA, 25 bp, Artificial Sequence
  Primer
ggattattgttaaatagtgtttttt

SEQ ID NO: 175 - DNA, 26 bp, Artificial Sequence
  Primer
aaccactctacattataactttttac

SEQ ID NO: 176 - DNA, 25 bp, Artificial Sequence
  Primer
ggattattgttaaatagtgtttttt

SEQ ID NO: 177 - DNA, 25 bp, Artificial Sequence
  Primer
atagttattttttaggtttgtgttt

SEQ ID NO: 178 - DNA, 25 bp, Artificial Sequence
  Primer
tatttagtgtgtgggaaagtgttga

SEQ ID NO: 179 - DNA, 25 bp, Artificial Sequence
  Primer
aaaatccaaacaaaaatccttacac

SEQ ID NO: 180 - DNA, 25 bp, Artificial Sequence
  Primer
agtattattgagggaatttttttag

SEQ ID NO: 181 - DNA, 25 bp, Artificial Sequence
  Primer
caactaatctaaaacatacaaaacc

SEQ ID NO: 182 - DNA, 25 bp, Artificial Sequence
  Primer
agtattattgagggaatttttttag

SEQ ID NO: 183 - DNA, 25 bp, Artificial Sequence
  Primer
gattttttggaggttgaatttggaa

SEQ ID NO: 184 - DNA, 25 bp, Artificial Sequence
  Primer
atttttttatttttttatttttttt

SEQ ID NO: 185 - DNA, 24 bp, Artificial Sequence
  Primer
atataacttttcctaatccaaaac

SEQ ID NO: 186 - DNA, 26 bp, Artificial Sequence
  Primer
tttggtaaattgtttaattttgtgga

SEQ ID NO: 187 - DNA, 27 bp, Artificial Sequence
  Primer
ttaacccacatatttaatcttccaatt

SEQ ID NO: 188 - DNA, 25 bp, Artificial Sequence
  Primer
tggattttgtagatggttttaaatg

SEQ ID NO: 189 - DNA, 25 bp, Artificial Sequence
  Primer
ggtagatattattgtataaattgtt

SEQ ID NO: 190 - DNA, 23 bp, Artificial Sequence
  Primer
tttttttaggtttaagtgaaaaa

SEQ ID NO: 191 - DNA, 25 bp, Artificial Sequence
  Primer
aatcctctattctaacaaaaataaa

SEQ ID NO: 192 - DNA, 25 bp, Artificial Sequence
  Primer
gtttgtttttttagggagtgtgtgt

SEQ ID NO: 193 - DNA, 30 bp, Artificial Sequence
  Primer
ccaaattctaataataattccttaaactct

SEQ ID NO: 194 - DNA, 25 bp, Artificial Sequence
  Primer
ggtaagtaaagaattgttatattat

SEQ ID NO: 195 - DNA, 25 bp, Artificial Sequence
  Primer
gttataatatttgttttgttattaa

SEQ ID NO: 196 - DNA, 26 bp, Artificial Sequence
  Primer
ttttttattttaaatagagtgattat

SEQ ID NO: 197 - DNA, 24 bp, Artificial Sequence
  Primer
tacaaataataaaacttaaaaccc

SEQ ID NO: 198 - DNA, 25 bp, Artificial Sequence
  Primer
tagttagattggaaagttagttagg

SEQ ID NO: 199 - DNA, 25 bp, Artificial Sequence
  Primer
tatactcaaaaaaataaaccaaaac

SEQ ID NO: 200 - DNA, 25 bp, Artificial Sequence
  Primer
tagttagattggaaagttagttagg

SEQ ID NO: 201 - DNA, 25 bp, Artificial Sequence
  Primer
ggaaggttttatagagtaattagag

SEQ ID NO: 202 - DNA, 25 bp, Artificial Sequence
  Primer
taatttttaagggtttgtgttataa

SEQ ID NO: 203 - DNA, 25 bp, Artificial Sequence
  Primer
atatgtatagtttgattgggtgtat

SEQ ID NO: 204 - DNA, 30 bp, Artificial Sequence
  Primer
aaaattatctttaaaaacataaaaaaataa

SEQ ID NO: 205 - DNA, 25 bp, Artificial Sequence
  Primer
tttgttttaaagtagggtttttttt

SEQ ID NO: 206 - DNA, 25 bp, Artificial Sequence
  Primer
aaactaatccaaactaccctcaatc

SEQ ID NO: 207 - DNA, 25 bp, Artificial Sequence
  Primer
ttttaaagtagggttttttttttag

SEQ ID NO: 208 - DNA, 25 bp, Artificial Sequence
  Primer
ttatagttatgggtggtgggggtgg

SEQ ID NO: 209 - DNA, 30 bp, Artificial Sequence
  Primer
gtgatagttataattatttagatgaataga

SEQ ID NO: 210 - DNA, 25 bp, Artificial Sequence
  Primer
aaaactttaacctacaaatatcccc

SEQ ID NO: 211 - DNA, 30 bp, Artificial Sequence
  Primer
ttggtatattatttatttttattaaaatag

SEQ ID NO: 212 - DNA, 25 bp, Artificial Sequence
  Primer
ataccaatatctttaatttcttcac

SEQ ID NO: 213 - DNA, 30 bp, Artificial Sequence; identical to SEQ ID NO: 211
  Primer

SEQ ID NO: 214 - DNA, 25 bp, Artificial Sequence
  Primer
tattaaaatagttttattaaattga

SEQ ID NO: 215 - DNA, 25 bp, Artificial Sequence
  Primer
tattttttgggattttgtttttttt

SEQ ID NO: 216 - DNA, 25 bp, Artificial Sequence
  Primer
tatgtagtttgttagttgttttttt

SEQ ID NO: 217 - DNA, 25 bp, Artificial Sequence
  Primer
ttgtgtgtgatagaaatttttgtat

SEQ ID NO: 218 - DNA, 25 bp, Artificial Sequence
  Primer
actaaaacaaccctaaatccatacc

SEQ ID NO: 219 - DNA, 27 bp, Artificial Sequence
  Primer
tgagggtatagagtttagtagaaattg

SEQ ID NO: 220 - DNA, 25 bp, Artificial Sequence
  Primer
cacaaaacaaacaaaaaaacaaatc

SEQ ID NO: 221 - DNA, 25 bp, Artificial Sequence
  Primer
tgagggtatagagtttagtagaaat

SEQ ID NO: 222 - DNA, 24 bp, Artificial Sequence
  Primer
gtttgttgtttttttttgtagtta

SEQ ID NO: 223 - DNA, 25 bp, Artificial Sequence
  Primer
gatttgtgatttttttgttaattta

(19) United States
(12) Patent Application Publication
(10) Pub. No.: US 2013/0281304 A1
(43) Pub. Date: Oct. 24, 2013

(54) Comprehensive Methylome Map of Myeloid and Lymphoid Commitment from Hematopoietic Proenitors

(71) Applicants: Andrew P. Feinberg, Lutherville, MD (US); Irving L. Weissman, Stanford, CA (US)

(72) Inventors: Andrew P. Feinberg, Lutherville, MD (US); Irving L. Weissman, Stanford, CA (US)

(21) Appl. No.: 13/814,460

(22) PCT Filed: Aug. 12, 2011
(86) PCT No.: PCT/US11/47681

§ 371 Date: Jul. 10, 2013

(30) Foreign Application Priority Data
Aug. 13, 2010 (US) 61373730

(51) Int. Cl. C12Q 1/68

(52) U.S. Cl. CPC C12Q 1/6881
USPC 506/2; 435/6.11; 435/377; 435/455

(57) ABSTRACT

Provided herein are differentially methylated regions (DMRs) of multipotent progenitor cells (MPPs) and oligopotent progenitor cells and methods of use thereof. The invention provides methods for detecting and analyzing alterations in the methylation status of DMRs in such progenitor cells as well as methods for differentiating such cells.

### STATEMENT OF GOVERNMENT SUPPORT

This invention was made with government support under R37CA053458, P50HG003233, R01AI047457, R01AI047458, and R00AGO29760 awarded by The National Institutes of Health. The United States government has certain rights in this invention.

### BACKGROUND OF THE INVENTION

1. Field of the Invention

The present invention relates generally to differentially methylated regions (DMRs) in the genome and more specifically to methods for detecting and analyzing alterations in the methylation status of DMRs in cells.

2. Background Information

Epigenetics is the study of non-sequence information of chromosome DNA during cell division and differentiation. The molecular basis of epigenetics is complex and involves modifications of the activation or inactivation of certain genes. Additionally, the chromatin proteins associated with DNA may be activated or silenced. Epigenetic changes are preserved when cells divide. Most epigenetic changes only occur within the course of one individual organism's lifetime, but some epigenetic changes are inherited from one generation to the next.

One example of an epigenetic mechanism is DNA methylation (DNAm), a covalent modification of the nucleotide cytosine. In particular, it involves the addition of methyl groups to cytosine nucleotides in the DNA, to convert cytosine to 5-methylcytosine. DNA methylation plays an important role in determining whether some genes are expressed or not.

Epigenetic modifications underlie lineage-specific differentiation as terminally differentiated cells express tissue-specific genes, but their DNA sequence is unchanged. Hematopoiesis provides a well-defined model to study epigenetic modifications during cell-fate fate decisions, as multipotent progenitors (MPPs) differentiate into progressively restricted myeloid or lymphoid progenitors. While DNA methylation is critical for myeloid versus lymphoid differentiation, as demonstrated by the myeloerythroid bias in Dnmt1 hypomorphs, a comprehensive DNA methylation map of hematopoietic progenitors, or of any multipotent/oligopotent lineage, does not exist.

Hematopoietic stem cells (HSC) can self renew for life and differentiate into all myeloid and lymphoid blood lineages (FIG. 1a). Recent evidence suggests that DNA methylation plays a direct role in regulating both HSC self-renewal and commitment to lymphoid versus myeloid fates. Although the frequencies of myeloid progenitors and differentiated cells were normal in Dnmt1-hypomorphic mice, lymphoid-restricted CLPs and their downstream thymic T cell progenitors (DN1, DN2 and DN3) were diminished. In the bone marrow of Dnmt1-hypomorphs, lymphoid, but not myeloid, transcripts were reduced, and promoters of two myeloerythroid genes were hypomethylated in HSCs.

These observations support a critical role for DNA methylation in lymphocyte development, possibly through regulation of gene expression.

### SUMMARY OF THE INVENTION

The present invention is based in part on the discovery of different epigenetic modifications associated with lineage-specific differentiation. Epigenetic modifications underlie lineage-specific differentiation as terminally differentiated cells express tissue-specific genes, but their DNA sequence is unchanged. Hematopoiesis provides a well-defined model to study epigenetic modifications during cell-fate decisions, as multipotent progenitors (MPPs) differentiate into progressively restricted myeloid or lymphoid progenitors.

Accordingly, in one aspect, the present invention provides a method of identifying the differentiation potential of a cell. The method includes comparing the methylation status of one or more nucleic acid sequences of a cell to a known methylation status of the one or more nucleic acid sequences of a reference progenitor cell. A similarity or a difference in methylation status between the cell and the reference cell is indicative of the differentiation potential of the cell. The method may further be performed with the proviso that the one or more nucleic acid sequences are outside of a promoter region of a gene and outside of a CpG island, and wherein the nucleic acid sequences are up to about 2 kb in distance from a CpG island. In some embodiments, the one or more nucleic acid sequences are selected from the group consisting of differentially methylated region (DMR) sequences as set forth in Tables 2a to 2h, FIGS. 1, 3, 5, 8, 9, 10 and any combination thereof.

In another aspect, the present invention provides a method of modifying the lineage restriction of a partially or terminally differentiated myeloid or lymphoid cell. The method includes contacting a partially or terminally differentiated myeloid or lymphoid cell with an agent which alters regulation of the expression or expression product of a gene known to be associated with the differentiation potential of the cell, thereby modifying the lineage restriction of the cell. In some embodiments, the agent alters regulation of the expression or expression product of a gene set forth in Tables 2a to 2h, Tables 3a to 3h, FIGS. 1, 3, 5, 8, 9, 10 and any combination thereof. In some embodiments the agent is a demethylating agent, such as a DNA (cytosine-5)-methyltransferase 1 (DNMT1) inhibitor, DNA (cytosine-5)-methyltransferase 1 (DNMT1) inhibitor, 5-azacytidine, 5-aza-2-deoxycytidine, or zebularine. In some embodiments, the agent is a vector comprising a nucleic acid sequence encoding a gene or portion thereof; a polynucleotide, polypeptide, or small molecule; an antisense oligonucleotide; or RNA, such as microRNA, dsRNA, siRNA, stRNA, or shRNA.

In another aspect, the present invention provides a method of inducing myeloid differentiation of a progenitor cell. The method includes contacting a progenitor cell with a demethylating agent, thereby inducing myeloid differentiation of the progenitor cell.

In another aspect, the present invention provides a method of differentiating a progenitor cell. The method includes contacting a progenitor cell with an agent that alters regulation of the expression or expression product of a gene known to be associated with the differentiation potential of the cell, thereby differentiating the progenitor cell. In some embodiments, the agent alters regulation of the expression or expression produce to a gene set forth in Tables 2a to 2h, Tables 3a to 3h, FIGS. 1, 3, 5, 8, 9, 10 and any combination thereof. In some embodiments, the expression of at least one of Meis1, Prdm16, 2900052L18Rik, H1f, Hoxa9, or Hoxa6, is decreases as compared to expression before contacting the cell with the agent.

In another aspect, the present invention provides a cell produced using the method described herein.

In another aspect, the present invention provides a population of cells, cell bank or library produced using the method described herein.

In another aspect, the present invention provides a method of treating a subject by introducing into the subject a cell produced by the method described herein.

In another aspect, the present invention provides a method of characterizing the methylation status of the nucleic acid of a cell. The method includes: a) hybridizing labeled and digested nucleic acid of an iPS cell to a DNA microarray comprising at least 2000 nucleic acid sequences; and b) determining a pattern of methylation from the hybridizing of (a), thereby characterizing the methylation status for the iPS cell. In various embodiments, the or more nucleic acid sequences are selected from the Tables 2a to 2h, Tables 3a to 3h, FIGS. 1, 3, 5, 8, 9, 10 and any combination thereof. The method may further include comparing the methylation status profile to a methylation profile from hybridization of the microarray with labeled and digested nucleic acid from a progenitor cell. In some embodiments the method may further include performing one or more techniques such as a nucleic acid amplification, polymerase chain reaction (PCR), methylation specific PCR, bisulfite pyrosequencing, single-strand conformation polymorphism (SSCP) analysis, and restriction analysis.

### BRIEF DESCRIPTION OF THE DRAWINGS

FIG. 1 is a series of graphical representations of known lineage-related genes showing differential DNA methylation between lymphoid and myeloid progenitors. FIG. 1a is a pictorial representation of the hematopoietic progenitors analyzed in the Example. Dashed-arrow indicates existence of intermediate progenitors. FIG. 1b are graphical representations of analysis of DMRs in Lck. FIG. 1c are graphical representations of analysis of DMRs in Mpo. Upper panels: top half: CpG methylation (p); lower half: CpG dinucleotides (black tick marks), CpG density (curve), CpG islands (grey lines) and the gene annotation (see Methods of Examples). Middle panels: methylation of individual CpGs (in the boxes), mean values connected by lines. Bottom panels: mRNA expression levels, normalized to the highest expression among the populations (mean±s.d., n=3;5 for MPP^{FL-} for microarrays).

FIG. 2 is a series of graphical representations of gene expression correlation with DMRs. DMRs within 2 kb of gene TSSs (black circles) were divided into two groups: Island (inside, cover, or overlap more than 50% of a CpG island), and Shores (up to 2000 bp away from a CpG island). After RMA preprocessing, the log2 ratios of the gene expression differences (from left to right) were plotted against Δp (left group minus right group). Black pluses represent random DMR-gene pairs more than 2 kb apart. Wilcoxon rank-sum tests were performed to test the null hypothesis. FIG. 2a plots MPP^{FL-} vs. DN3_DMRs. FIG. 2b plots MPP^{FL-} vs.GMP_DMRs.

FIG. 3 is a series of graphical representations of CHARM plots, pyrosequencing, Affymetrix GeneChip, and RT-PCR data of CHARM identified genes with previously unknown functions in lymphoid/myeloid lineage commitment and pluripotency maintenance. FIG. 3a depicts the DMR in Arl4c. FIG. 3b depicts the DMR in Jdp2. FIG. 3c depicts the DMR in Meis1. FIG. 3d depicts the DMR in Hdac7a.

FIG. 4 is a series of graphical representations of purification of progenitor populations using FACS. FIG. 4a is a series of FACS plots of progenitor populations purified from mouse bone-marrow cells. Selection was based on the combination of cell-surface marker expressions defined as follows: MPP^{FL-}, Lin⁻I17ra⁻c-Kit⁺Sca-1⁺CD34⁺Flk2; MPP^{FL+}, Lin⁻I17ra⁻c-Kit⁺Sca-1⁺CD34⁺Flk2⁺; CMP, Lin⁻I17ra⁻c-Kit⁺Sca-1⁻CD34⁺FcyRII/III^{low}, GMP: Lin⁻I17ra⁻c-Kit⁺Sca-1⁻CD34⁺FcyRII/III⁺. FIG. 4b is a series of FACS plots of progenitor populations purified from mouse bone-marrow cells. Selection was based on the combination of cell-surface marker expressions defined as follows: CLP, Lin⁻CD27⁺Flk2⁺I1ra⁺Ly6D⁻. FIG. 4c is a series of FACS plots of progenitor populations purified from mouse thymocyte. Selection was based on the combination of cell-surface marker expressions defined as follows: DN1, Lin⁻CD4⁻CD8⁻c-Kit⁺CD44⁺CD25⁻; DN2, Lin⁻CD4⁻CD8⁻c-Kit⁺CD44⁺CD25⁺; DN3, Lin⁻CD4⁻CD8⁻c-Kit⁻CD44⁻CD25⁺.

FIG. 5 is a series of graphical representations of CHARM plots, pyrosequencing, and Affymetrix GeneChip data of known lineage-related genes identified to be differentially methylated between lymphoid and myeloid progenitors. FIG. 5a is the DMR in Cxcr2. FIG. 5b is of the DMR in Gadd45a.

FIG. 6 is a series of graphical representations of FACS plots and quantitation of myeloid versus lymphoid progeny. FIG. 6a are representative FACS plots of progeny from hematopoietic progenitors 6 days after plating on OP9:OP9DL1 stroma in the presence or absence of 5-aza-2′deoxycytidine. With the addition of 5-aza-2′deoxycytidine, MPP^{FL+}, CLP, DN1, and DN2 trended towards generating more myeloid progeny, while DN3 continued to generate an exclusively lymphoid readout. Limited readout from DN3 wells is due to inhibited cell expansion by 5-aza-2′deoxycytidine. FIG. 6b is a histogram depicting quantitation of myeloid versus lymphoid progeny. The percentage of myeloid versus lymphoid progeny, as defined above, were quantified for each well from the experiment in a. Each bar represents the average of triplicate wells, and the error bars are the standard deviations between wells.

FIG. 7 is a series of graphical representations showing gene expression correlation with DMRs. DMRs within 2 kb of gene TSSs (black circles) were divided into two groups: Island (inside, cover, or overlap more than 50% of a CpG island) and Shores (>0 and <=2000 bp away from a CpG island). After RMA preprocessing, the log₂ ratios of the gene expression differences (from left to right) were plotted against Δp (left group minus right group). Black pluses represent random DMR-gene pairs more than 2 kb apart. Wilcoxon rank-sum tests were performed to test the null hypothesis that the distribution of expression differences for the hypomethylated (or hypermethylated) DMRs (black circles) is shifted higher (or lower) than the distribution of expression differences for the black pluses. FIG. 7a plots MPP^{FL-} vs. MPP^{FL+}_DMRs. FIG. 7b plots MPP^{FL+} vs. CMP_DMRs. FIG. 7c plots MPP^{FL+} vs. CLP_DMRs. FIG. 7d plots CLP vs. DN1_DMRs. FIG. 7e plots DN1 vs. DN2_DMRs. FIG. 7f plots DN2 vs. DN3_DMRs. FIG. 7g plots MPP^{FL-} vs. DN3_DMRs_{—}2 (CHARM 1.1). FIG. 7h plots MPP^{FL-} vs. GMP_DMRs_{—}2 (CHARM 1.1).

FIG. 8 is a series of graphical representations of CHARM plots, pyrosequencing, Affymetrix GeneChip, and RT-PCR data. FIG. 8a is of DMR in Smad7. FIG. 8b is of DMR in Gcnt2. FIG. 8c is of DMR in Cited2. FIG. 8d is of DMR in Dach1.

FIG. 9 is a series of graphical representations of CHARM plots, pyrosequencing, Affymetrix GeneChip, and RT-PCR data. FIG. 9a is of DMR in 2900052L18Rik. FIG. 9b is of DMR in Hlf. FIG. 9c is of DMR in Hoxa9. FIG. 9d is of DMR in Prdm16.

FIG. 10 is a series of graphical representations of CHARM plots, pyrosequencing, and Affymetrix GeneChip data. FIG. 9a is of DMR in Dnmt3b.

### DETAILED DESCRIPTION OF THE INVENTION

The present invention is based in part on the discovery of different epigenetic alterations associated with lineage-specific differentiation in cells ranging from uncommitted MPPs through oligopotent progenitors specified during myeloid versus lymphoid fate decisions. Hematopoiesis provides a well-defined model to study epigenetic modifications during cell-fate decisions, as MPPs differentiate into progressively restricted myeloid or lymphoid progenitors.

Hematopoiesis is the formation of blood cells from hematopoietic stem cells (HSC), a population of multipotent cells that can self renew for life and differentiate into all myeloid and lymphoid lineages. HSCs can differentiate into multipotent progenitors (MPPs), which retain multipotency but not self-renewal capacity. Two oligopotent progenitors derive from MPPs; the common lymphoid progenitor (CLP) and the common myeloid progenitor (CMP). During myeloid-lineage differentiation, CMPs give rise to megakaryocyte-erythrocyte progenitors (MEPs) and granulocyte-macrophage progenitors (GMPs), which progressively differentiate to yield all mature myeloid and erythroid cell types, such as monocytes, neutrophils, basophils, eosinophils, erythrocytes, and megakaryocytes. CLPs further differentiate through lymphoid-restricted intermediates to generate all mature lymphoid cells, such as T cells, B cells, and natural killer cells.

While DNA methylation is critical for myeloid versus lymphoid differentiation, as demonstrated by the myeloerythroid bias in Dnmt1 hypomorphs, a comprehensive DNA methylation map of hematopoietic progenitors, or of any multipotent/oligopotent lineage, does not exist. In the Example provided herein, 4.6 million CpG sites were examined throughout the genome for MPPs, common lymphoid progenitors (CLPs), common myeloid progenitors (CMPs), granulocyte/macrophage progenitors (GMPs), and thymocyte progenitors (DN1, DN2, DN3). Dramatic epigenetic plasticity accompanied both lymphoid and myeloid restriction. Myeloid commitment involved less global DNA methylation than lymphoid commitment, supported functionally by myeloid skewing of progenitors following treatment with a DNA methyltransferase inhibitor. Differential DNA methylation correlated with gene expression more strongly at CpG island shores than CpG islands. Many examples of genes and pathways not previously known to be involved in choice between lymphoid/myeloid differentiation have been identified, such as Arl4c and Jdp2. Several transcription factors, including Meis1, were methylated and silenced during differentiation, suggesting a role in maintaining an undifferentiated state. Additionally, epigenetic modification of modifiers of the epigenome appears to be important in hematopoietic differentiation.

The examples provided herein directly demonstrate that modulation of DNA methylation occurs during lineage-specific differentiation and defines a comprehensive map of the methylation and transcriptional changes that accompany myeloid versus lymphoid fate decisions.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

In accordance with this discovery, there are provided herein differentially methylated regions (DMRs) of multipotent progenitor cells (MPPs) and oligopotent progenitor cells, as well as methods of use thereof. The invention provides methods for detecting and analyzing alterations in the methylation status of DMRs in such progenitor cells as well as methods for differentiating such cells.

Cellular differentiation is the process by which a less specialized cell becomes a more specialized cell type, and is accompanied by dramatic changes in cellular characteristics, such as cell size, shape, membrane potential, metabolic activity, and responsiveness to signals. These changes are largely due to highly-controlled modifications in gene expression. Cell differentiation is thus a transition of a cell from one cell type to another and typically involves a switch from one pattern of gene expression to another.

Accordingly, in one aspect of the invention, there is provided a method of identifying the differentiation potential of a cell. The method includes comparing the methylation status of one or more nucleic acid sequences of a cell to a known methylation status of the one or more nucleic acid sequences of a reference progenitor cell. A similarity or a difference in methylation status between the cell and the reference cell is indicative of the differentiation potential of the cell. The method may further be performed with the proviso that the one or more nucleic acid sequences are outside of a promoter region of a gene and outside of a CpG island, and wherein the nucleic acid sequences are up to about 2 kb in distance from a CpG island.

In another aspect, the present invention provides a method of modifying the lineage restriction of a partially or terminally differentiated myeloid or lymphoid cell. The method includes contacting a partially or terminally differentiated myeloid or lymphoid cell with an agent which alters regulation of the expression or expression product of a gene known to be associated with the differentiation potential of the cell, thereby modifying the lineage restriction of the cell.

Modifying the lineage restriction of a cell may include inducing differentiation of the cells into a more differentiated state or “reprogramming” the cell to a less differentiated state. As used herein, “differentiation” refers to a change that occurs in cells to cause those cells to assume certain specialized functions and to lose the ability to change into certain other specialized functional units. Cells capable of differentiation may be any of totipotent, pluripotent or multipotent cells. Differentiation may be partial or complete with respect to mature adult cells.

As used herein, “reprogramming”, is intended to refer to a process that alters or reverses the differentiation status of a cell that is either partially or terminally differentiated, such as a myeloid or lymphoid cell. Reprogramming of a cell may be a partial or complete reversion of the differentiation status of the cell. In one aspect, reprogramming is complete wherein a partially or terminally differentiated cell is reprogrammed into an induced pluripotent stem (iPS) cell. However, reprogramming may be partial, such as reversion into any less differentiated state. For example, reverting a terminally differentiated cell, such as a restricted myeloid or lymphoid progenitor cell into a cell of a less differentiated state, such as a multipotent cell.

As used herein, pluripotent cells include cells that have the potential to divide in vitro for an extended period of time (greater than one year) and have the unique ability to differentiate into cells derived from progenitor cells.

As used herein, “multipotent” or “multipotent cell” refers to a cell type that can give rise to a limited number of other particular cell types.

In another aspect, the present invention provides a method of inducing myeloid differentiation of a progenitor cell. The method includes contacting a progenitor cell with a demethylating agent, thereby inducing myeloid differentiation of the progenitor cell.

In another aspect, the present invention provides a method of differentiating a progenitor cell. The method includes contacting a progenitor cell with an agent that alters regulation of the expression or expression product of a gene known to be associated with the differentiation potential of the cell, thereby differentiating the progenitor cell.

As discussed herein, alterations in methylation patterns occur during differentiation or dedifferention of a cell which work to regulate gene expression of critical factors that are ‘turned on’ or ‘turned off’ at various stages of differentiation. As such, one of skill in the art would appreciate that many types of agents are capable of altering the methylation status of one or more nucleic acid sequences of a cell to either dedifferentiate or further differentiate a cell that may be suitable for use with the present invention.

As discussed in the Example, DMRs and genes that have been determined to be associated with the differentiation potential of MPPs, common lymphoid progenitors (CLPs), common myeloid progenitors (CMPs), granulocyte/macrophage progenitors (GMPs), and thymocyte progenitors (DN1, DN2, DN3).

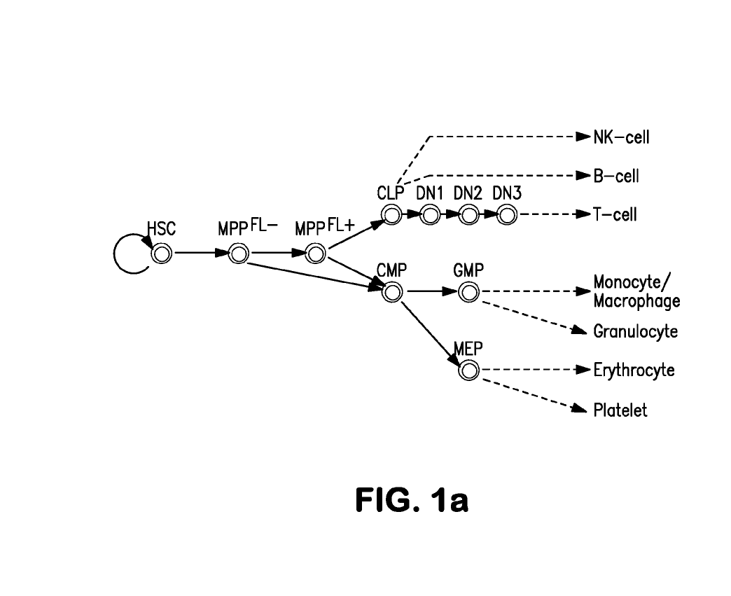

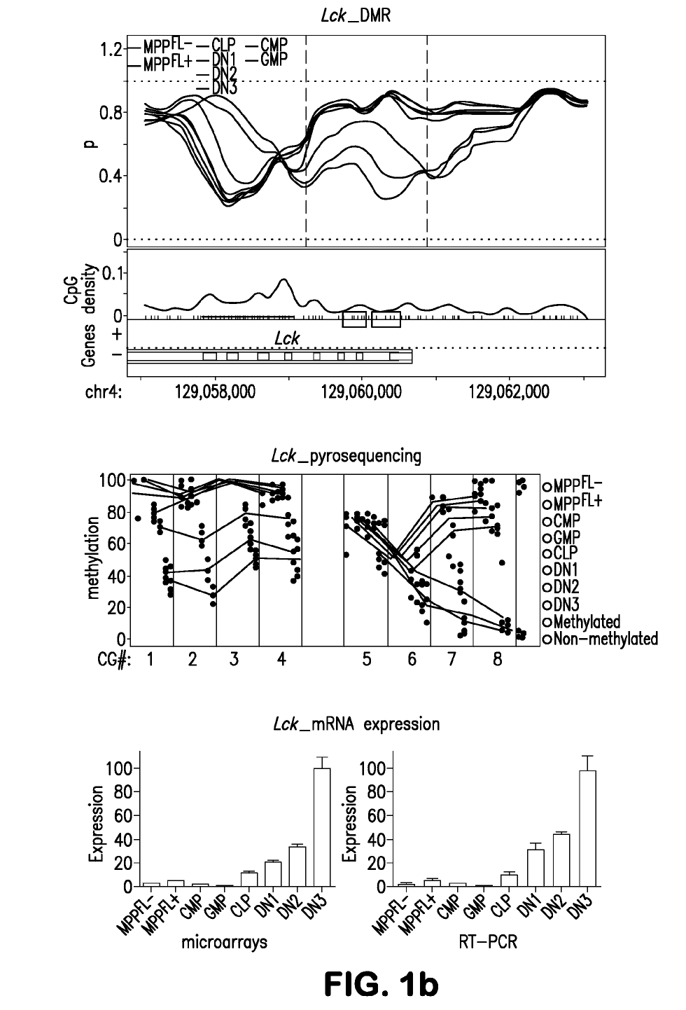

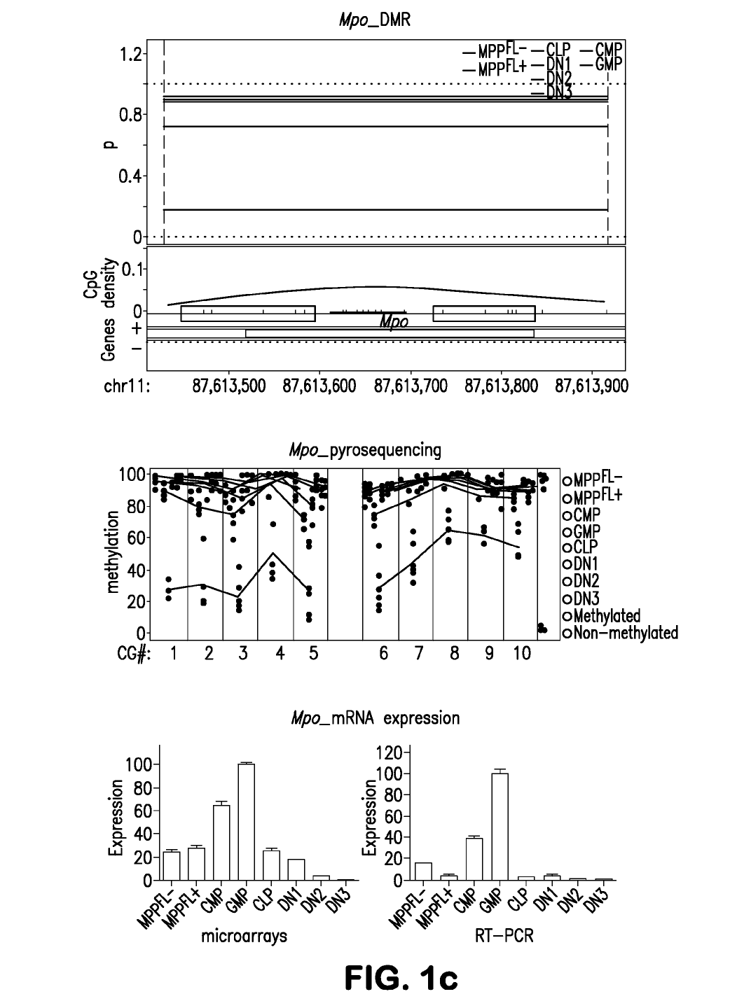

Accordingly, an agent as used herein, is intended to include any agent capable of altering the methylation status of one or more nucleic acid sequences, such as DMR sequences as set forth in Tables 2a to 2h, FIGS. 1, 3, 5, 8, 9, 10 and any combination thereof. Additionally, an agent as used hrein, is intended to include any agent capable of altering regulation of the expression or expression product of a gene set forth in Tables 2a to 2h, Tables 3a to 3h, FIGS. 1, 3, 5, 8, 9, 10 and any combination thereof.

An agent useful in any method of the invention may be any type of molecule, for example, a polynucleotide, a peptide, a peptidomimetic, peptoids such as vinylogous peptoids, chemical compounds, such as organic molecules or small organic molecules, or the like. In various aspects, the agent may be a polynucleotide, such as DNA molecule, an antisense oligonucleotide or RNA molecule, such, as microRNA, dsRNA, siRNA, stRNA, and shRNA.

MicroRNA (miRNA) are single-stranded RNA molecules whose expression is known to be regulated by methylation to play a key role in regulation of gene expression during differentiation and dedifferentiation of cells. Thus an agent may be one that inhibits or induces expression of miRNA or may be a mimic miRNA. As used herein, “mimic” microRNAs which are intended to mean a microRNA exogenously introduced into a cell that have the same or substantially the same function as their endogenous counterpart.

In various aspects of the present invention, an agent that alters the methylation status of one or more nucleic acid sequences may be reprogramming factors or differentiation factors. Reprogramming factors may be genes that induce pluripotency and utilized to reprogram differentiated or semi-differentiated cells to a phenotype that is more primitive than that of the initial cell, such as the phenotype of a MPP. Those skilled in the art would understand that such genes and agents are capable of generating a cell that is less differentiated upon expression of one or more such genes having been integrated into the genome of the cell or upon contact of the cell with the agent or expression product of the gene. As used herein, a reprogramming factor may be a gene that is associated with generating a less differentiated cell, for example a MPP from a further differentiated cell, such as a progressively restricted myeloid or lymphoid cell, upon integration and expression of the gene.

Differentation factors may be genes that induce pluripotency and utilized to direct differentiation of a cell to a phenotype that is more more lineage restricted than that of the initial cell. Those skilled in the art would understand that such genes and agents are capable of generating a cell that is more differentiated upon expression of one or more such genes having been integrated into the genome of the cell or upon contact of the cell with the agent or expression product of the gene. As used herein, a differentiaton factor may be a gene that is associated with generating a more differentiated cell, for example a common myeloid progenitor from a MPP, upon integration and expression of the gene.

Several genes have been found to be associated with the differentiation potential of progenitor cells as well as lineage restriction of myeloid and lymphoid cells which are suitable for use with the present invention. Such genes are set forth in Tables 2a to 2h, Tables 3a to 3h, as well as FIGS. 1, 3, 5, 8, 9, 10.

All of these genes commonly exist in mammals, including human, and thus homologues from any mammals may be used in the present invention, such as genes derived from mammals including, but not limited to mouse, rat, bovine, ovine, horse, and ape. Further, in addition to wild-type gene products, mutant gene products including substitution, insertion, and/or deletion of several (e.g., 1 to 10, 1 to 6, 1 to 4, 1 to 3, and 1 or 2) amino acids and having similar function to that of the wild-type gene products can also be used. Furthermore, the combinations of factors are not limited to the use of wild-type genes or gene products.

The present invention is not limited to any particular combination of reprogramming and differentiation factors. As discussed herein a reprogramming or differentiation factor may comprise one or more gene products. The reprogramming or differentiation factor may also comprise a combination of gene products as discussed herein. Each reprogramming or differentiation factor may be used alone or in combination with other factors as disclosed herein.

The reprogramming or differentiation factor may include a protein or peptide. The protein may be produced from a gene as discussed herein, or alternatively, in the form of a fusion gene product of the protein with another protein, peptide or the like. The protein or peptide may be a fluorescent protein and/or a fusion protein. For example, a fusion protein with green fluorescence protein (GFP) or a fusion gene product with a peptide such as a histidine tag can also be used. Further, by preparing and using a fusion protein with the TAT peptide derived from the virus HIV, intracellular uptake of the nuclear reprogramming factor through cell membranes can be promoted, thereby enabling induction of reprogramming only by adding the fusion protein to a medium thus avoiding complicated operations such as gene transduction. Since preparation methods of such fusion gene products are well known to those skilled in the art, skilled artisans can easily design and prepare an appropriate fusion gene product depending on the purpose.

In various embodiments, the agent may commonly be known as a demthylating agent. As used herein, the term “demethylating agent” is used to refer to any compound that can inhibit methylation, resulting in the expression of the previously hypermethylated silenced genes. Cytidine analogs such as 5-azacytidine (azacitidine) and 5-aza-2-deoxycytidine (decitabine) are the most commonly used demethylating agents. These compounds work by binding to the enzymes that catalyze the methylation reaction, DNA methyltransferases. Thus, in one embodiment, the demethylating agent is 5-azacytidine, 5-aza-2-deoxycytidine, or zebularine. In another embodiment, the demethylating agent may be an inhibitor of DNA (cytosine-5)-methyltransferase 1 (DNMT1).

Detecting the methylation status profile of the one or more nucleic acid sequences of the cell contacted with the agent or factor and/or comparing the methylation status profile to a methylation status profile of the one or more nucleic acid sequences of a parental or daughter cell from which the cell is induced may also be performed to assess pluripotency characteristics.

Similarly, expression profiling of lineage restriction altered cells to assess their pluripotency characteristics may also be conducted. Expression of individual genes associated with lineage restriction may also be examined. Additionally, expression of cell surface markers may be analyzed. As used herein, “expression” refers to the production of a material or substance as well as the level or amount of production of a material or substance. Thus, determining the expression of a specific marker refers to detecting either the relative or absolute amount of the marker that is expressed or simply detecting the presence or absence of the marker. As used herein, “marker” refers to any molecule that can be observed or detected. For example, a marker can include, but is not limited to, a nucleic acid, such as a transcript of a specific gene, a polypeptide product of a gene, a non-gene product polypeptide, a glycoprotein, a carbohydrate, a glycolipd, a lipid, a lipoprotein or a small molecule.

Detection and analysis of a variety of genes known in the art to be associated with hematopoetic stem (HPS) cells and differentiated cells therefrom may include analysis of genes such as, but not limited to CD34, SCA-1, CD59, Thy1, CD38, C-kit and lin. Additional markers for detection may include a cluster of differentiation (CD) molecule cell, including but not limited to CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, and CD66e.

The invention further provides cells whose lineage has been altered using the methods described herein, as well as populations of such cells. The cells of the present invention, have a variety of applications and therapeutic uses. For example, the basic properties of progenitor cells are the capability to infinitely self-renew and the ability to differentiate into a variety of cell types in the body make them ideal for therapeutic uses.

Accordingly, in one aspect the present invention further provides a method of treatment or prevention of a disorder and/or condition in a subject using cells generated using the methods described herein. The method includes obtaining a cell from a subject and altering the differentiation potential of the cell using the methods described herein. In one embodiment, the cell is then cultured under suitable conditions to further differentiate the cell into a desired cell type suitable for treating a condition. The differentiated cell may then be introducing into the subject to treat or prevent the condition. In another embodiment, the cell is then cultured under suitable conditions to further de-differentiate the cell into a cell of a less differentiated state suitable for treating a condition. The de-differentiated cell may then be introducing into the subject to treat or prevent the condition.

One advantage of the present invention is that it provides an essentially limitless supply of isogenic or synegenic human cells suitable for transplantation. The cells are tailored specifically to the patient, avoiding immune rejection. Therefore, it will obviate the significant problem associated with current transplantation methods, such as, rejection of the transplanted tissue which may occur because of host versus graft or graft versus host rejection. Several kinds of de-differentiated cells or fully differentiated cells prepared from healthy humans can be stored in a cell bank as a library of cells, and one kind or more kinds of the cells in the library can be used for preparation of cells that are free of rejection by a patient to be subjected to such cell therapy.

The de-differentiated cells of the present invention may be differentiated into a number of different cell types to treat a variety of disorders by methods known in the art. For example, progenitor cells may be induced to differentiate into myeloid and lympoid derived cells, which include a variety of blood cells. The differentiated cells may then be transplanted back into the patient's body to prevent or treat a condition.

In various embodiments, the cell is administered directly to a subject at a site where an increase in cell number is desired either before or after differentiation of the cell to a desired cell type.

Methylome analysis allows for the characterization of cells by analyzing DMR patterns. As such, the present invention provides a method of characterizing the methylation status of the nucleic acid of a cell. The method includes a) hybridizing labeled and digested nucleic acid of a cell to a DNA microarray comprising at least 2000 nucleic acid sequences; b) determining a pattern of methylation from the hybridizing of (a), thereby characterizing the methylation status for the cell, wherein the one or more nucleic acid sequences are selected from those set forth in Tables 2a to 2h, Tables 3a to 3h, FIGS. 1, 3, 5, 8, 9, 10 and any combination thereof.

The invention further provides a plurality of nucleic acid sequences, wherein wherein the plurality of nucleic acid sequences are selected from those set forth in Tables 2a to 2h, Tables 3a to 3h, FIGS. 1, 3, 5, 8, 9, 10 and any combination thereof.

In various embodiments of the invention, the plurality of nucleic acid sequences may be utilized to provide a microarray for performing the methods described herein. One skilled in the art would appreciate the many techniques that are well known for attaching nucleic acids on a substrate that may be utilized along with the various types of substrates and configurations.

Characterizing the methylation status of the nucleic acid of an iPS cell may further include comparing the methylation status profile to a methylation profile from hybridization of the microarray with labeled and digested nucleic acid from a cell from which the cell is induced or differentiated from, or from an MPP cell. In particular embodiments, the one or more nucleic acid sequences are DMR sequences as set forth in Tables 2a to 2h, FIGS. 1, 3, 5, 8, 9, 10 and any combination thereof.

In various aspects of the invention, methylation status is converted to an M value. As used herein an M value, can be a log ratio of intensities from total (Cy3) and McrBC-fractionated DNA (Cy5): positive and negative M values are quantitatively associated with methylated and unmethylated sites, respectively.

In various aspects of the invention DMR may be hypermethylated or hypomethylated. Hypomethylation of a DMR is present when there is a measurable decrease in methylation of the DMR. In some embodiments, a DMR can be determined to be hypomethylated when less than 50% of the methylation sites analyzed are not methylated. Hypermethylation of a DMR is present when there is a measurable increase in methylation of the DMR. In some embodiments, a DMR can be determined to be hypermethylated when more than 50% of the methylation sites analyzed are methylated. Methods for determining methylation states are provided herein and are known in the art. In some embodiments methylation status is converted to an M value. As used herein an M value, can be a log ratio of intensities from total (Cy3) and McrBC-fractionated DNA (Cy5): positive and negative M values are quantitatively associated with methylated and unmethylated sites, respectively. M values are calculated as described in the Examples. In some embodiments, M values which range from −0.5 to 0.5 represent unmethylated sites as defined by the control probes, and values from 0.5 to 1.5 represent baseline levels of methylation.

Numerous methods for analyzing methylation status of a gene are known in the art and can be used in the methods of the present invention to identify either hypomethylation or hypermethylation of the one or more DMRs. In various embodiments, the determining of methylation status in the methods of the invention is performed by one or more techniques selected from the group consisting of a nucleic acid amplification, polymerase chain reaction (PCR), methylation specific PCR, bisulfite pyrosequenceing, single-strand conformation polymorphism (SSCP) analysis, restriction analysis, microarray technology, and proteomics. As illustrated in the Examples herein, analysis of methylation can be performed by bisulfite genomic sequencing. Bisulfite treatment modifies DNA converting unmethylated, but not methylated, cytosines to uracil. Bisulfite treatment can be carried out using the METHYLEASY bisulfite modification kit (Human Genetic Signatures).

In some embodiments, bisulfite pyrosequencing, which is a sequencing-based analysis of DNA methylation that quantitatively measures multiple, consecutive CpG sites individually with high accuracy and reproducibility, may be used. Exemplary primers for such analysis are set forth in the Examples as well as Table 4.

It will be recognized that depending on the site bound by the primer and the direction of extension from a primer, that the primers listed above can be used in different pairs. Furthermore, it will be recognized that additional primers can be identified within the DMRs, especially primers that allow analysis of the same methylation sites as those analyzed with primers that correspond to the primers disclosed herein.

Altered methylation can be identified by identifying a detectable difference in methylation. For example, hypomethylation can be determined by identifying whether after bisulfite treatment a uracil or a cytosine is present a particular location. If uracil is present after bisulfite treatment, then the residue is unmethylated. Hypomethylation is present when there is a measurable decrease in methylation.

In an alternative embodiment, the method for analyzing methylation of the DMR can include amplification using a primer pair specific for methylated residues within a DMR. In these embodiments, selective hybridization or binding of at least one of the primers is dependent on the methylation state of the target DNA sequence (Herman et al., Proc. Natl. Acad. Sci. USA, 93:9821 (1996)). For example, the amplification reaction can be preceded by bisulfite treatment, and the primers can selectively hybridize to target sequences in a manner that is dependent on bisulfite treatment. For example, one primer can selectively bind to a target sequence only when one or more base of the target sequence is altered by bisulfite treatment, thereby being specific for a methylated target sequence.

Other methods are known in the art for determining methylation status of a DMR, including, but not limited to, array-based methylation analysis and Southern blot analysis.

Methods using an amplification reaction, for example methods above for detecting hypomethylation or hyprrnethylation of one or more DMRs, can utilize a real-time detection amplification procedure. For example, the method can utilize molecular beacon technology (Tyagi et al., Nature Biotechnology, 14:303 (1996)) or Taqman™ technology (Holland et al., Proc. Natl. Acad. Sci. USA, 88:7276 (1991)).

Also methyl light (Trinh et al., Methods 25(4):456-62 (2001), incorporated herein in its entirety by reference), Methyl Heavy (Epigenomics, Berlin, Germany), or SNuPE (single nucleotide primer extension) (see e.g., Watson et al., Genet Res. 75(3):269-74 (2000)) Can be used in the methods of the present invention related to identifying altered methylation of DMRs.

As used herein, the term “selective hybridization” or “selectively hybridize” refers to hybridization under moderately stringent or highly stringent physiological conditions, which can distinguish related nucleotide sequences from unrelated nucleotide sequences.

As known in the art, in nucleic acid hybridization reactions, the conditions used to achieve a particular level of stringency will vary, depending on the nature of the nucleic acids being hybridized. For example, the length, degree of complementarity, nucleotide sequence composition (for example, relative GC:AT content), and nucleic acid type, for example, whether the oligonucleotide or the target nucleic acid sequence is DNA or RNA, can be considered in selecting hybridization conditions. An additional consideration is whether one of the nucleic acids is immobilized, for example, on a filter. Methods for selecting appropriate stringency conditions can be determined empirically or estimated using various formulas, and are well known in the art (see, e.g., Sambrook et al., supra, 1989).

An example of progressively higher stringency conditions is as follows: 2× SSC/0.1% SDS at about room temperature (hybridization conditions); 0.2× SSC/0.1% SDS at about room temperature (low stringency conditions); 0.2× SSC/0.1% SDS at about 42° C. (moderate stringency conditions); and 0.1× SSC at about 68° C. (high stringency conditions). Washing can be carried out using only one of these conditions, for example, high stringency conditions, or each of the conditions can be used, for example, for 10 to 15 minutes each, in the order listed above, repeating any or all of the steps listed.

The degree of methylation in the DNA associated with the DMRs being assessed, may be measured by fluorescent in situ hybridization (FISH) by means of probes which identify and differentiate between genomic DNAs, associated with the DMRs being assessed, which exhibit different degrees of DNA methylation. FISH is described, for example, in de Capoa et al. (Cytometry. 31:85-92, 1998) which is incorporated herein by reference. In this case, the biological sample will typically be any which contains sufficient whole cells or nuclei to perform short term culture. Usually, the sample will be a sample that contains 10 to 10,000, or, for example, 100 to 10,000, whole cells.

Additionally, as mentioned above, methyl light, methyl heavy, and array-based methylation analysis can be performed, by using bisulfite treated DNA that is then PCR-amplified, against microarrays of oligonucleotide target sequences with the various forms corresponding to unmethylated and methylated DNA.

The term “nucleic acid molecule” is used broadly herein to mean a sequence of deoxyribonucleotides or ribonucleotides that are linked together by a phosphodiester bond. As such, the term “nucleic acid molecule” is meant to include DNA and RNA, which can be single stranded or double stranded, as well as DNA/RNA hybrids. Furthermore, the term “nucleic acid molecule” as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR), and, in various embodiments, can contain nucleotide analogs or a backbone bond other than a phosphodiester bond.

The terms “polynucleotide” and “oligonucleotide” also are used herein to refer to nucleic acid molecules. Although no specific distinction from each other or from “nucleic acid molecule” is intended by the use of these terms, the term “polynucleotide” is used generally in reference to a nucleic acid molecule that encodes a polypeptide, or a peptide portion thereof, whereas the term “oligonucleotide” is used generally in reference to a nucleotide sequence useful as a probe, a PCR primer, an antisense molecule, or the like. Of course, it will be recognized that an “oligonucleotide” also can encode a peptide. As such, the different terms are used primarily for convenience of discussion.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

In another aspect, the present invention includes kits that are useful for carrying out the methods of the present invention. The components contained in the kit depend on a number of factors, including: the particular analytical technique used to detect methylation or measure the degree of methylation or a change in methylation, and the one or more DMRs is being assayed for methylation status.

Accordingly, the present invention provides a kit for determining a methylation status of one or more DMRs of the invention. In some embodiments, the one or more DMRs are selected from one or more of the sequences as set forth in Tables 2a to 2h, FIGS. 1, 3, 5, 8, 9, 10 and any combination thereof. The kit includes an oligonucleotide probe, primer, or primer pair, or combination thereof for carrying out a method for detecting hypomethylation, as discussed above. For example, the probe, primer, or primer pair, can be capable of selectively hybridizing to the DMR either with or without prior bisulfite treatment of the DMR. The kit can further include one or more detectable labels.

The kit can, also include a plurality of oligonucleotide probes, primers, or primer pairs, or combinations thereof, capable of selectively hybridizing to the DMR with or without prior bisulfite treatment of the DMR. The kit can include an oligonucleotide primer pair that hybridizes under stringent conditions to all or a portion of the DMR only after bisulfite treatment. In one aspect, the kit can provide reagents for bisulfite pyrosequencing including one or more primer pairs set forth in Tables 11 and 12. The kit can include instructions on using kit components to identify, for example, the presence of cancer or an increased risk of developing cancer.

To examine DNAm on a genome-wide scale, comprehensive high-throughput array-based relative methylation (CHARM) analysis, which is a microarray-based method agnostic to preconceptions about DNAm, including location relative to genes and CpG content was carried out. The resulting quantitative measurements of DNAm, denoted with M, are log ratios of intensities from total (Cy3) and McrBC-fractionated DNA (Cy5): positive and negative M values are quantitatively associated with methylated and unmethylated sites, respectively. For each sample, ˜4.6 million CpG sites across the genome of iPS cells, parental somatic cells and ES cells were analyzed using a custom-designed NimbleGen HD2 microarray, including all of the classically defined CpG islands as well as all nonrepetitive lower CpG density genomic regions of the genome. 4,500 control probes were included to standardize these M values so that unmethylated regions were associated, on average, with values of 0. CHARM is 100% specific at 90% sensitivity for known methylation marks identified by other methods (for example, in promoters) and includes the approximately half of the genome not identified by conventional region preselection. The CHARM results were also extensively corroborated by quantitative bisulfite pyrosequencing analysis.

Provided herein is a genome-wide analysis of DNA methylation addressing variation among various MPP cells and progressibely restricted myeloid and lympoid cells, revealing several surprising differences and relationships among the cell types of epigenetic variation, supported by extensive bisulfite pyrosequencing and functional analysis.

In one aspect of the invention, methylation density is determined for a region of nucleic acid, for example any region identified in Tables 2a to 2h, Tables 3a to 3h, FIGS. 1, 3, 5, 8, 9 and 10. Density may be used as an indication of the differentiation potential of a cell, for example. A density of about 0.2 to 0.7, about 0.3 to 0.7 , 0.3 to 0.6 or 0.3 to 0.4, or 0.3, may be indicative of generation of particular lineage restricted cell type (the calculated DNA methylation density is the number of methylated CpGs divided by the total number of CpGs sequenced for each sample). Methods for determining methylation density are well known in the art. For example, a method for determining methylation density of target CpG islands has been established by Luo et al. (Analytical Biochemistry, 387:2 2009, pp. 143-149). In the method, DNA microarray was prepared by spotting a set of PCR products amplified from bisulfite-converted sample DNAs. This method not only allows the quantitative analysis of regional methylation density of a set of given genes but also could provide information of methylation density for a large amount of clinical samples as well as use in the methods of the invention regarding iPS cell generation and detection. Other methods are well known in the art (e.g., Holemon et al., BioTechniques, 43:5, 2007, pp. 683-693).

The following examples are provided to further illustrate the advantages and features of the present invention, but are not intended to limit the scope of the invention. While they are typical of those that might be used, other procedures, methodologies, or techniques known to those skilled in the art may alternatively be used.

### EXAMPLE 1

### Differential Methylation of Myeloid and Lymphoid Commitment

The following experimental methods and protocols were utilized to analyze and identify DMRs in numerous genes throughout the genome which play a role in lymphoid or myeloid fate specification.

Flow cytometry. Bone marrow cells and thymocytes were stained with monoclonal antibodies, then analyzed and sorted on a FACSAria (Beckton Dickinson, San Jose, Calif.). The following monoclonal antibodies were purified and conjugated using hybridomas maintained in the Weissman laboratory: anti-CD8 (53.6.7) conjugated to Alexa Fluor™488, anti-CD4 (GK1.5) conjugated to Alexa Fluor 647, anti-CD44 (IM781) conjugated to Alexa Fluor™680, anti-CD25 (PC.61) conjugated to PacificOrange™, anti-FcγRII/III (2.4G2) conjugated to PacificOrange™, anti-Sca-1 (E13-161-7) conjugated to Pacific Blue™, and anti-Ly6D (49H4.3, courtesy provided by Herzenberg laboratory, Stanford) conjugated to PacificOrange™. The following antibodies were purchased from eBioscience (San Diego, Calif.): anti-CD34 (RAM34) conjugated to FITC; anti-C135/F1k2 (A2F10) conjugated to PE; anti-CD127/I17ra (A7R34) conjugated to PE-Cy5; anti-CD4 (GK1.5), -CD8 (53-6.7), -B220 (RA3-6B2), -Ter119 (TER119), -Mac-1 (M1/70), and anti-Gr-1 (RB6-8C5) conjugated to PE-Cy7; anti-CD27 (LG.7F9) conjugated to APC, anti-c-Kit (2B8) conjugated to APC-Alexa Fluor™750.

CHARM DNA methylation analysis. Genomic DNA from each sample was purified using the MasterPure DNA™ purification kit (Epicentre) as recommended by the manufacturer. 1.5˜2ug genomic DNA was fractionated, digested with McrBC, gel purified, labeled and hybridized to a CHARM microarray as described. CHARM microarrays (CHARM 1.0) are prepared as previously described using custom-designed Nimblegen HD2™ microarrays (Irizarry et al., Genome Res. 2008;18:780-790). For the new CHARM arrays used in this study (CHARM 1.1), ˜11% of probes with lowest CpG density on CHARM 1.0 were substituted with probes in promoters that weren't previously covered. For each probe, the average methylation values across the same cell type were computed and converted to the percentage of methylation (p). p was used to find regions of differential methylation (Δp) for each pairwise cell type comparison. The absolute area of each region was calculated by multiplying the number of probes by mean Δp and DMRs were ranked based on this absolute area. In the CHARM plots, the upper panel shows the extent of methylation across a region of the genome. The top half of the panel is a plot of the percentage of CpG methylation versus genomic location, where the curve represents averaged smoothed p values from each cell population indicated (four replicates of MPP^{FL-} and MPP^{FL+} and three replicates of the remaining cell populations; 40,000-100,000 cells of each population were sorted for each replicate). Two vertical dotted lines mark the range of the DMR identified. The lower half of the panel illustrates the location of CpG dinucleotides (black tick marks), CpG density (curve), location of CpG islands (grey line) and the gene annotation. + or − on the left side of the bottom panel indicates the orientation of genes and grey boxes represent exons with numbers indicated. Detailed information of DMRs identified in this study is listed in Table 4. CHARM microarray data are deposited at the Gene Expression Omnibus (ncbi.nlm.nih.gov/geo) under accession number GSE23110.

Bisulfite pyrosequencing. 200 ng of genomic DNA from each sample (6 samples for each progenitor) was treated with bisulfite using an EZ DNA methylation-Gold Kit™ (ZYMO research) according to the manufacturer's specifications. The bisulfite-treated genomic DNA was amplified by PCR using unbiased nested primers and DNA methylation was measured by quantitative pyrosequencing using a PSQ HS96 (Biotage). The DNA methylation percentage at each CpG site was determined using the Q-CpG methylation software (Biotage). SssI treated mouse genomic DNA was used as 100% methylation control and mouse genomic DNA amplified by GenomePlex Complete. Whole Genome Amplification (WGA) Kit™ (Sigma) was used as the non-methylated DNA control. Primer sequences used for the bisulfite pyrosequencing reactions are shown in Table 4, as well as the chromosomal coordinates in the University of California at Santa Cruz March 2006 mouse genome assembly for each CpG site measured. The annealing temperature used for all PCR reactions was between 50˜55 ° C.

GO annotation. GO annotation was analyzed using NIA Mouse Gene Index (lgsun.grc.nia.nih.gov/geneindex/mm9/upload.html). Genes identified from the analysis were compared to genes on arrays to calculate the enrichment ratio and significantly enriched gene ontology functional categories (FDR<0.05) are included in Table 3.

Affymetrix microarray expression analysis. Genome-wide gene expression analysis was performed using Affymetrix GeneChip Mouse Genome 430 2.0 Array™. For each sample, 1 μg of high-quality total RNA was amplified, labeled and hybridized onto the microarray according to Affymetrix's specifications, and data were normalized by GC-RMA method and analyzed on R/Bioconductor (GEO accession number GSE20244).

OP9:OP9DL1 stromal co-cultures. 3000 OP9 and 3000 OP9DL1 cells were plated in each well of 96 well plates in MEMa+10% FBS. The next day, 50 double-sorted progenitors were plated per well in the presence of 5 ng/ml IL-7 and F1t3L, and 10 ng/ml IL-3, IL-6, M-CSF, GM-CSF, and G-CSF (PeproTech). 50 nM 5-aza-2′-deoxycytide (Sigma) or vehicle (50% acetic acid) was added to the wells as indicated. At day 3, half of the media plus cytokines and drugs was replaced. At day 6, progeny from each well were stained and analyzed by flow cytometry to identify lymphoid versus myeloid progeny.

Quantitative PCR. Cells were sorted into TRIzol (Invitrogen Life Technologies, Carlsbad, Calif.), and RNA was isolated according to manufacturer's instruction. cDNA was synthesized using the Superscript III™ kit (Invitrogen Life Technologies) using random hexamers. Amplifications were performed using SYBR Green PCR™ core reagents (Applied Biosystems), and transcript levels were quantified using an ABI 7900 Sequence Detection Systems™ (Applied Biosystems). Mean Ct value of triplicate reaction was normalized against mean Ct value of beta-actin. Amplification efficiency of each primer pair was validated prior application using cDNA libraries of mouse ES cells, whole BM cells, and whole spleen cells. Primers sequences are as follows:

β-actin:
(SEQ ID NO: 1)
5′-GTCTGAGGCCTCCCTTTTT-3′
and
(SEQ ID NO: 2)
5′-GGGAGACCAAAGCCTTCATA-3′.
Lck:
(SEQ ID NO: 3)
5′-TGGAGAACATTGACGTGTGTG-3′;
and
(SEQ ID NO: 4)
5′-ATCCCTCATAGGTGACCAGTG-3′.
Mpo:
(SEQ ID NO: 5)
5′-CCACGGAGCTCCTGTTTTAC-3′;
and
(SEQ ID NO: 6)
5′-CAGCTTCCTCTTCAGCAGGT-3′.
Gcnt2:
(SEQ ID NO: 7)
5′-TGCTCATCTTTCATCGACGGA-3′;
and
(SEQ ID NO: 8)
5′-AGTGGCTTTGGGTCACATATTC-3′.
Arl4c:
(SEQ ID NO: 9)
5′-AGTCTCTGCACATCGTTATGC-3′;
and
(SEQ ID NO: 10)
5′-GGTGTTGAAGCCGATAGTGGG-3′.
Dach1:
(SEQ ID NO: 11)
5′-CCTGGGAAACCCGTGTACTC-3′;
and
(SEQ ID NO: 12)
5′-AGATCCACCATTTTGCACTCATT-3′.
Jdp2:
(SEQ ID NO: 13)
5′-AGCTGAAATACGCTGACATCC-3′;
and
(SEQ ID NO: 14)
5′-CTCACTCTTCACGGGTTGGG-3′.
Meis1:
(SEQ ID NO: 15)
5′-CATGATAGACCAGTCCAACCGA-3′;
and
(SEQ ID NO: 16)
5′-ATTGGCTGTCCATCAGGGTTA-3′.
Prdm16:
(SEQ ID NO: 17)
5′-TGACGGATACAGAGGTGTCAT-3′;
and
(SEQ ID NO: 18)
5′-ACGCTACACGGATGTACTTGA-3′.
Dnmt3b:
(SEQ ID NO: 19)
5′-GTTAATGGGAACTTCAGTGACCA-3′;
and
(SEQ ID NO: 20)
5′-CTGCGTGTAATTCAGAAGGCT-3′.
Hdac7a:
(SEQ ID NO: 21)
5′-TTCCCTACAGAACTCTTGAGCC-3′;
and
(SEQ ID NO: 22)
5′-GGGGCACTCTCCTTCCTGA-3′.

DNA methylation query website. The following website, charm.jhmi.edu/hsc, allows plotting of DNA methylation in any region from the CHARM array. Regions of interest are uploaded as a tab or comma-separated file. Top50 DMRs plots from the complete sets are listed.

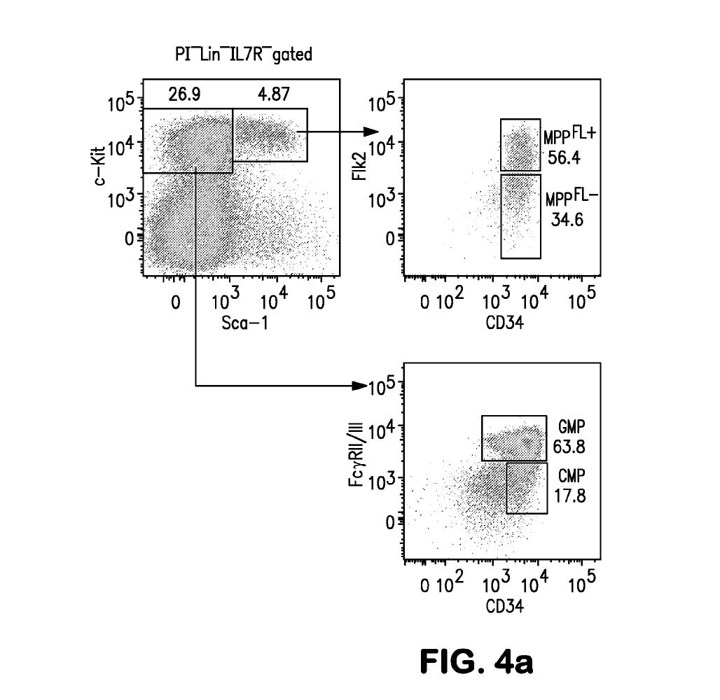

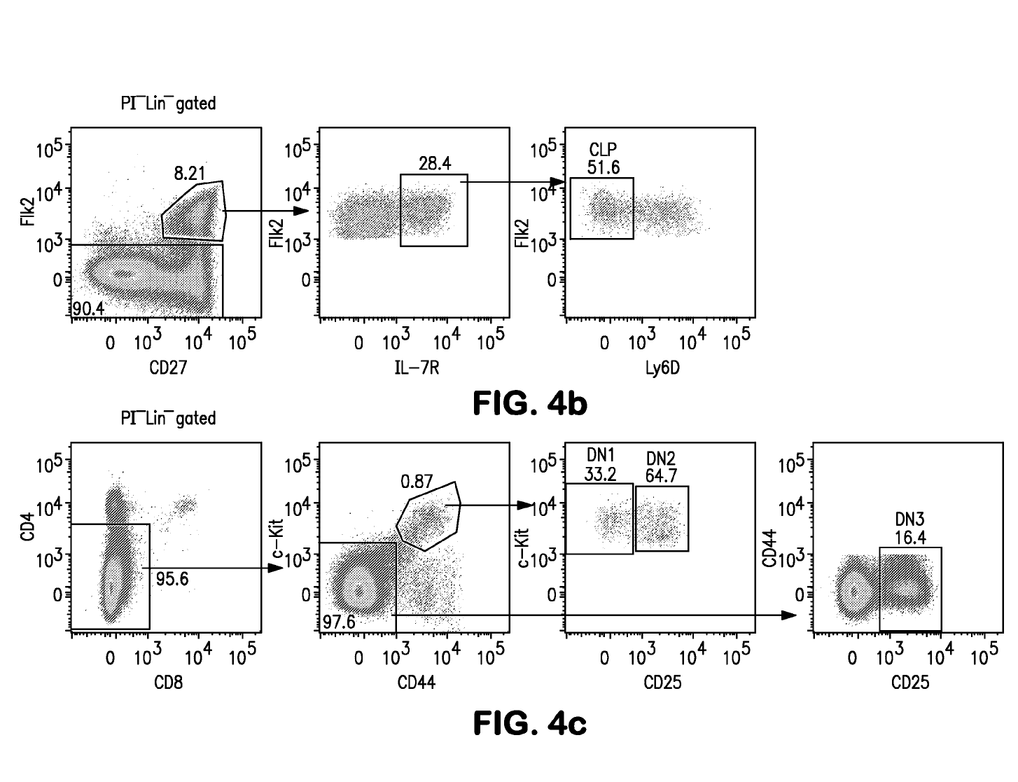

Genome-wide methylation profiles of the mouse hematopoietic system were examined, because it provides the first opportunity to examine differential methylation of a hierarchical progression of purified cell populations with well-characterized differentiation potentials (FIG. 1a). Eight populations, ranging from uncommitted MPP through oligopotent progenitors specified during myeloid versus lymphoid fate decisions, were FACS-purified and subjected to Comprehensive High-throughput Array-based Relative Methylation (CHARM) analysis (FIG. 1a and FIG. 4). Examples of known lineage-related genes showing differential DNA methylation between lymphoid and myeloid progenitors are shown in FIG. 1. FIG. 4 provides a prospective isolation strategy of progenitor populations using FACS. Progenitor populations were purified from mouse bone-marrow cells (FIG. 4a and FIG. 4b) or thymocyte (FIG. 4c) based on a combination of cell-surface marker expressions.

This approach investigated the methylation status of CpGs throughout the mouse genome using an algorithm favoring regions of higher CpG density (including all CpG islands (see, Gardiner-Garden et al., J Mol Biol. 1987;196:261-282)), but without bias for CpG location relative to genes (discussed in Irizarry et al., Genome Res. 2008;18:780-790). Using CHARM, it was recently determined that differential methylation occurs more frequently in CpG island “shores” (regions within 2 kb of an islands) than in CpG islands during multiple cellular differentiation processes Additionally, mRNA of each population was subjected to microarray and RT-PCR analyses to generate gene expression data. Thus, it was possible to directly compare differentially methylated regions (DMRs) throughout the genome with expression levels of nearby genes for all eight populations.

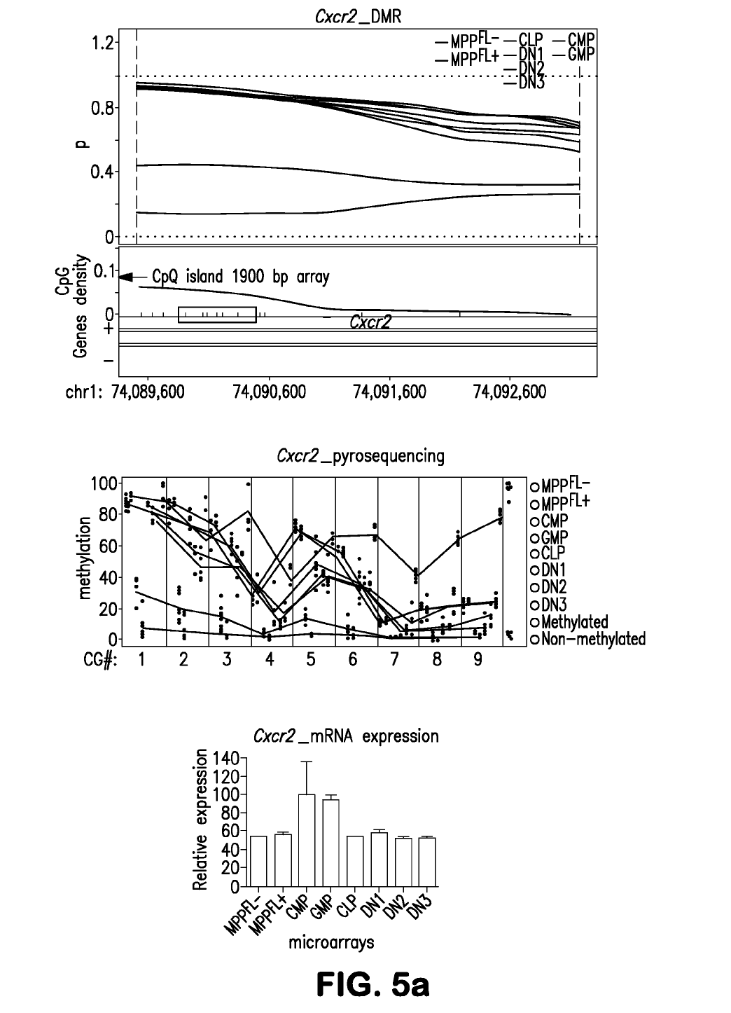

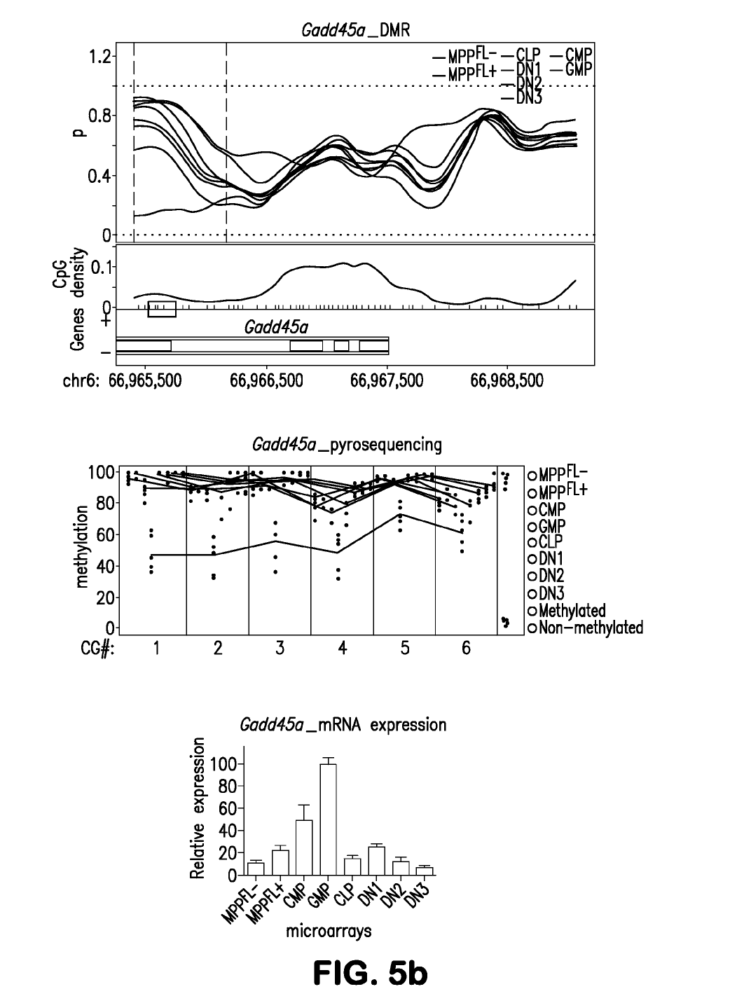

This analysis revealed DMRs in numerous genes known to play a role in lymphoid or myeloid fate specification. For example, Lck, the src family kinase member responsible for initiating signaling downstream of the T cell receptor (TCR), was transcriptionally upregulated from DN1 to DN3, consistent with its role in pre-TCR signal transduction (FIG. 1b). Interestingly, as Lck transcription was upregulated, CpGs in exon 1 through intron 2 were progressively demethylated (FIG. 1b). Similarly, myeloid specification from MPP through GMP was accompanied by transcriptional upregulation and progressive hypomethylation of Mpo, which encodes an enzyme central to the microbicidal activity of neutrophils (FIG. 1c). Additionally, Cxcr2, which encodes a chemokine receptor responsible for neutrophil chemotaxis, was upregulated during myeloid commitment from CMP through GMP, while the gene was demethylated (FIG. 5a). Furtheimore, Gadd45a, which is implicated in myeloid development, was found to be concomitantly upregulated and demethylated in the CMP to GMP transition (FIG. 5b).

Known lineage-related genes were identified to be differentially methylated between lymphoid and myeloid progenitors as shown in FIG. 5. The DMR in Cxcr2 is shown in FIG. 5a, and the DMR in Gadd45a is shown in FIG. 5b. In each case, the upper panel shows the extent of methylation across a region of the genome. The top half of the panel is a plot of the percentage of CpG methylation (p value; see Methods) versus genomic location, where the curve represents averaged smoothed p values from MPP^{FL-}, MPP^{FL+}, CMP, GMP, CLP, DN1, DN2, and DN3 samples. Four replicates of MPP^{FL-} and MPP^{FL+}, and three replicates of the remaining cell populations were assayed. Two vertical dotted lines mark the range of the DMR identified. The lower half of the panel illustrates the location of CpG dinucleotides (black tick marks), CpG density (curve), location of CpG islands (grey line) and the gene annotation. The orientations of the genes is indicated by + or − on the left side of the bottom panel and grey boxes represent exons. The middle panels show methylation of individual CpG sites measured by bisulfite pyrosequencing (mapped to the red boxes in the upper panels and labeled with the same colors as in the CHARM plots). Colored dots represent the percentage of methylation from a separate set of 6 samples for each progenitor, as well as the ˜100% methylated DNA treated by SssI methyltransferase in vitro and low methylated DNA after whole genome amplification. Lines were drawn to connect the mean methylation values of each progenitor at the interrogated CpG sites. Nine CpG sites in Cxcr2 and six CpG sites in Gadd45a were assayed quantitatively. The bottom panels show mRNA expression levels of Cxcr2 from Affymetrix GeneChip microarrays for each progenitor (labeled with the same color as in CHARM plots). Bars represent the expression level for each population relative to the highest expression among the populations (mean expression ±s.d., n=3 (5 for MPP^{FL-})).

Gadd45a can actively demethylate DNA in different model systems; thus, hypomethylation of Gadd45a during myelopoiesis may promote further hypomethylation of genes regulating myeloid commitment; however, the role of Gadd45a in promoting demethylation is still controversial. Taken together, these data indicate that CHARM analysis correctly identifies DMRs in known lymphoid and myeloid specifying genes, each confirmed by pyrosequencing and gene expression analysis, making it a valuable tool for identifying candidate genes important for lymphoid or myeloid fate specification.

Viewed globally, CHARM analysis revealed striking epigenetic plasticity, resulting in increased overall methylation upon lymphoid relative to myeloid commitment (Table 1). Most DMRs distinguishing MPP^{FL+} cells from CLP lost methylation during this step of early lymphoid commitment, but upon the subsequent transition to DN1, 15-fold more DMRs showed gain, as opposed to loss, of methylation. Similarly in the earliest step of myeloid commitment from MPP^{FL+} to CMP there were substantially more hypermethylated than hypomethylated DMRs, but nearly all DMRs showed loss of methylation on transition from CMP to GMP. Comparing DN1 to GMP, two populations similarly differentiated towards lymphoid and myeloid fates, respectively, there were 8-fold more DMRs with higher-level methylation in DN1 cells, suggesting a skewing toward greater methylation in lymphoid compared to myeloid hematopoiesis. These observations might explain why Dnmt1-hypomorphic mice, which are unable to properly maintain CpG methylation, have normal myeloid, but diminished lymphoid development.

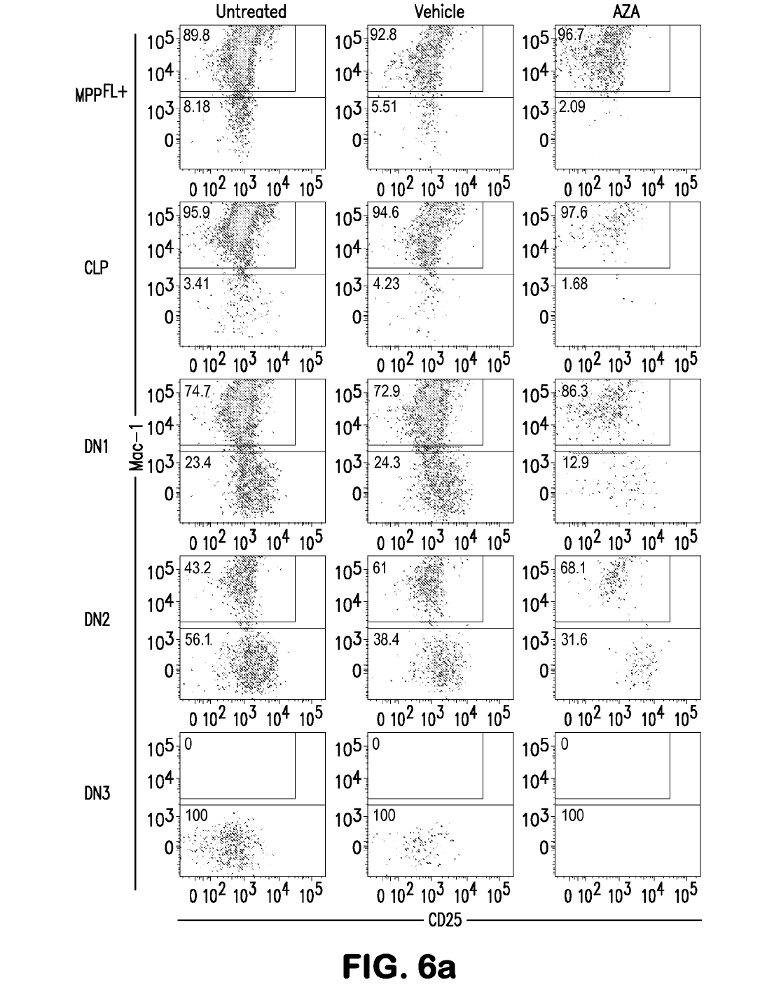

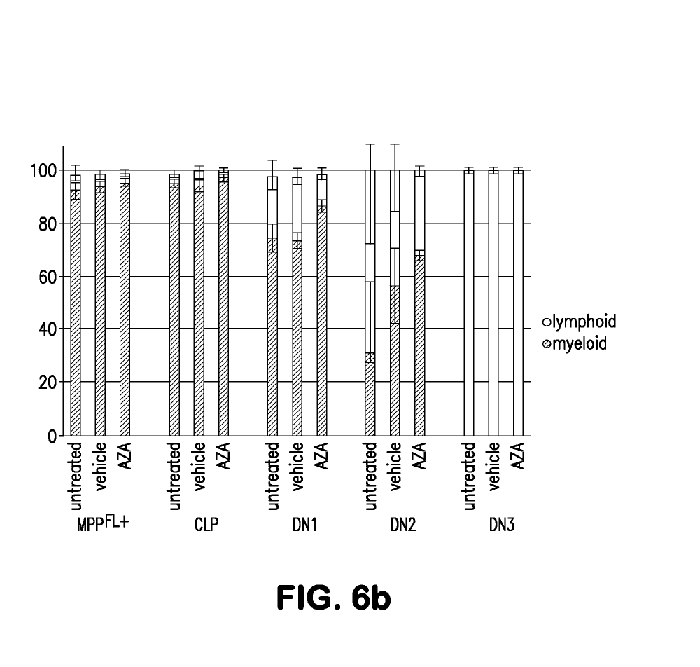

To test the hypothesis that reduced methylation preferentially promotes myeloid as opposed to lymphoid differentiation, an in vitro assay system that promotes both myeloid and lymphoid development was used (as in Engel et al., Epigenetics. 2009;4:98-99 and Bell et al., Nature. 2008;452:764-767). In the presence of 5-aza-2′-deoxycytidine, the percentage of myeloid progeny increased at the expense of lymphoid progeny for MPP^{FL+}, CLP, DN1 and DN2, but not DN3, which remained lymphoid committed (FIGS. 6a-b). This myeloid skewing was most pronounced in DN1 cells, perhaps indicating that the large number of methylated DMRs in DN1 compared to CLP is critical for lymphoid specification (Table 1). It evident that inhibiting DNA methylation promotes myeloid versus lymphoid specification, providing a mechanism for the myeloid skewing observed in Dnmt1 hypomorphs.

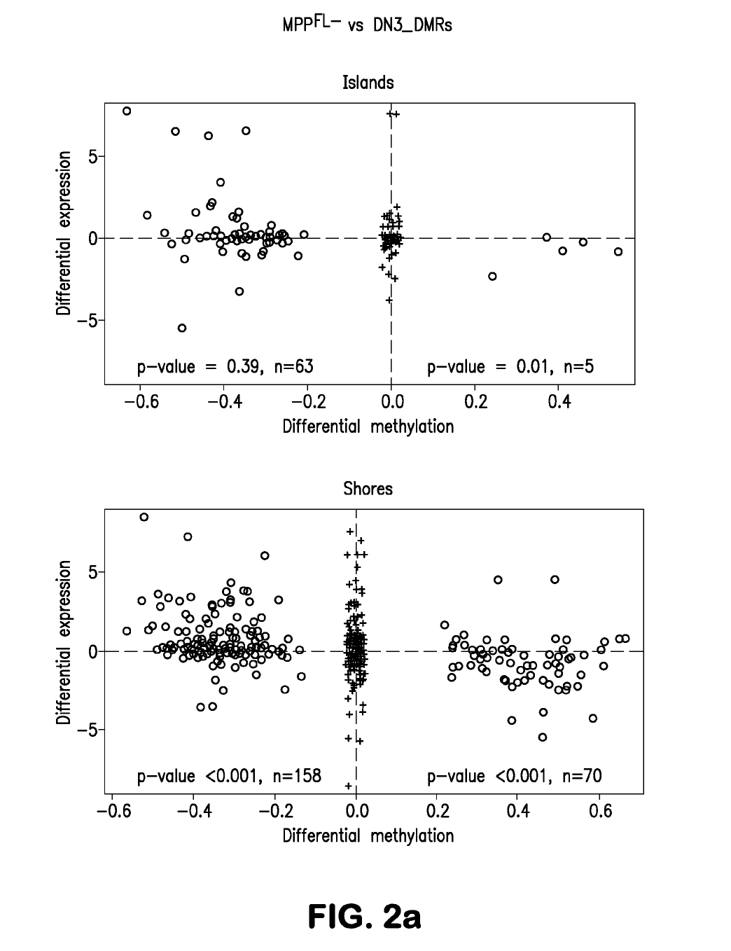

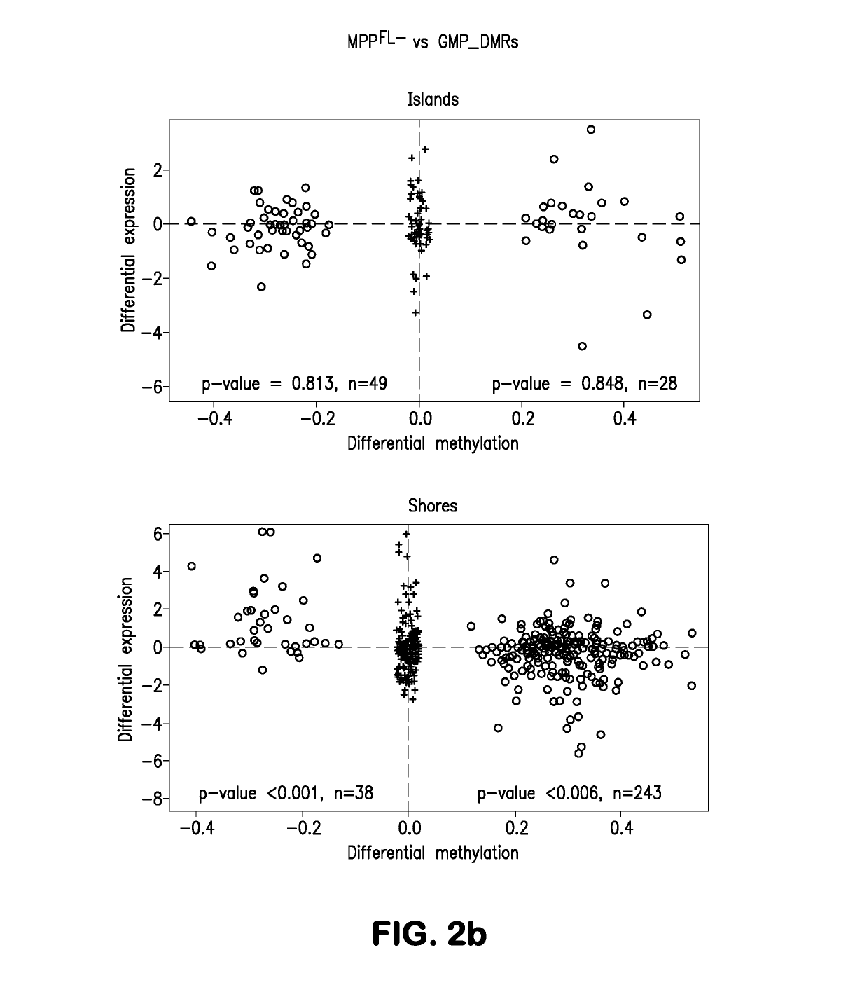

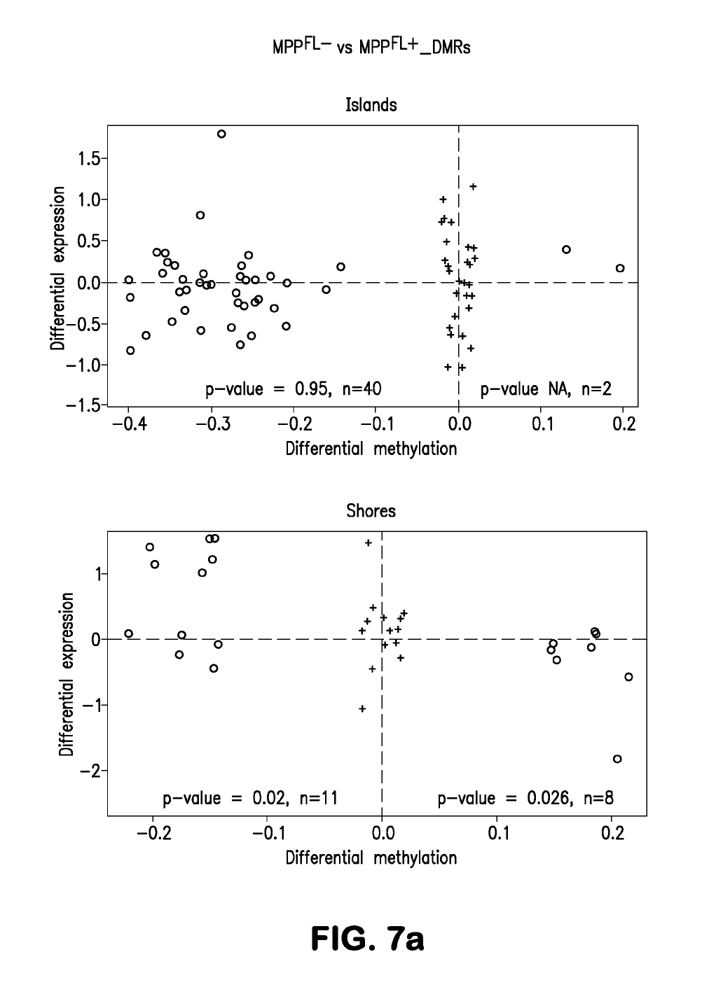

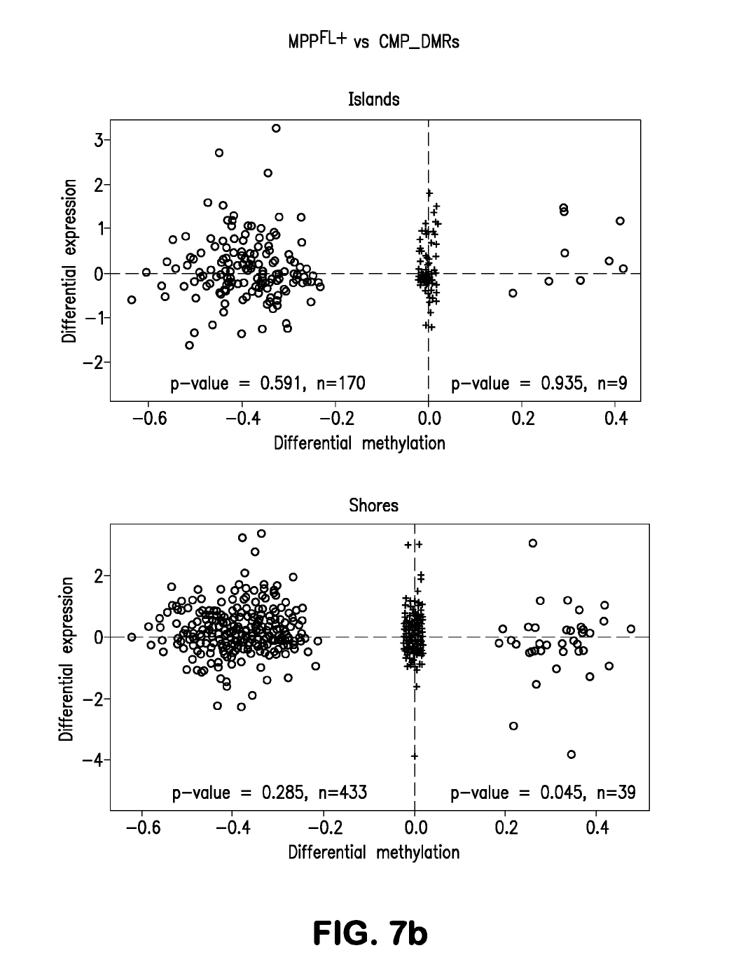

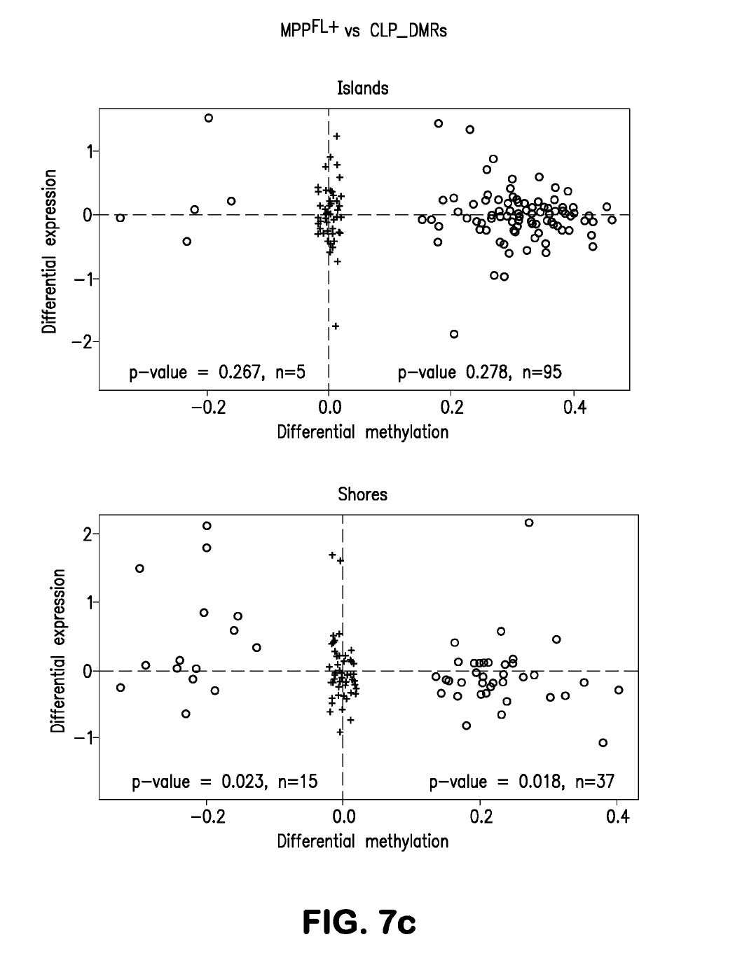

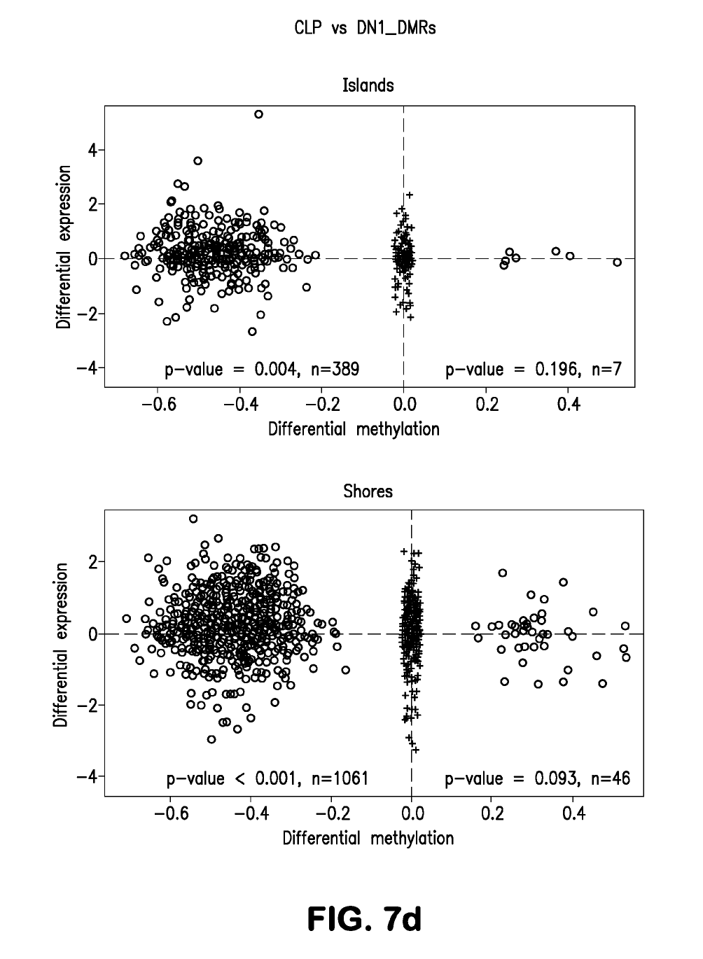

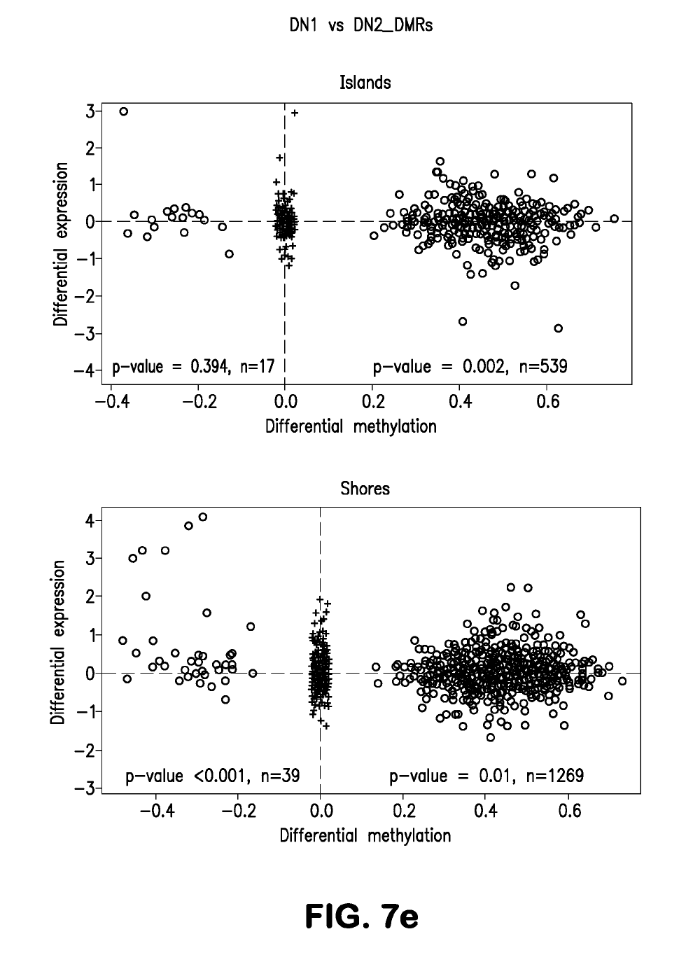

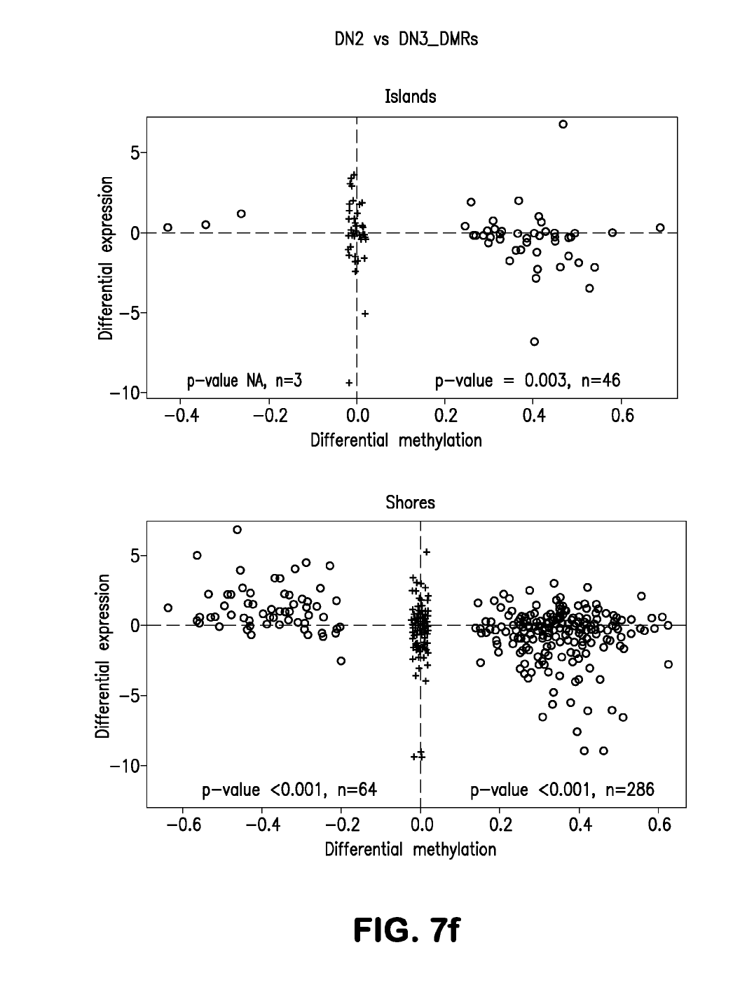

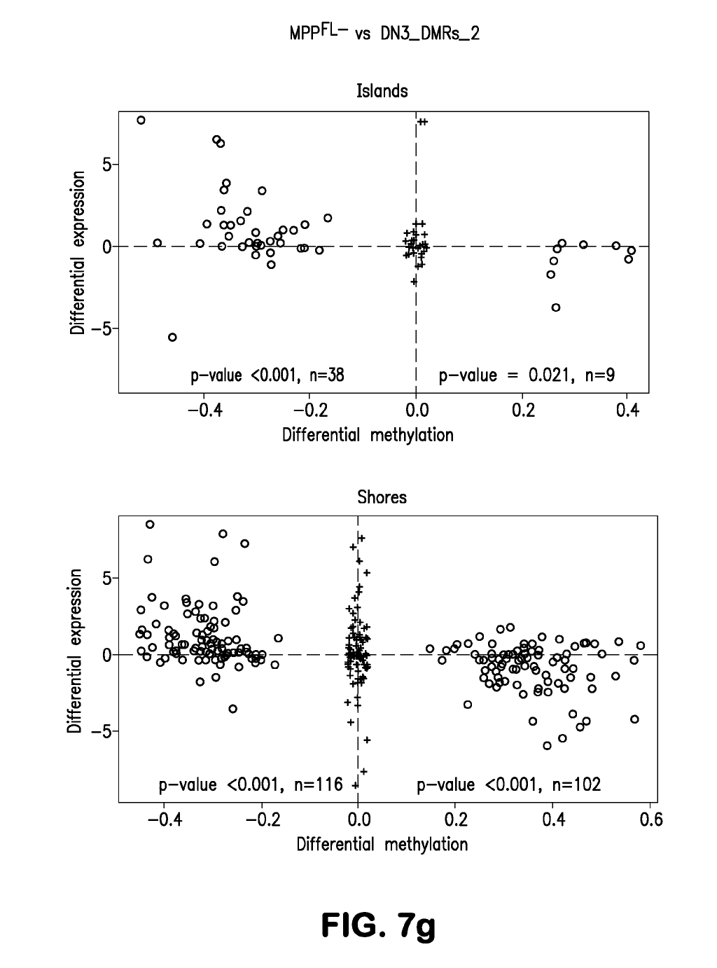

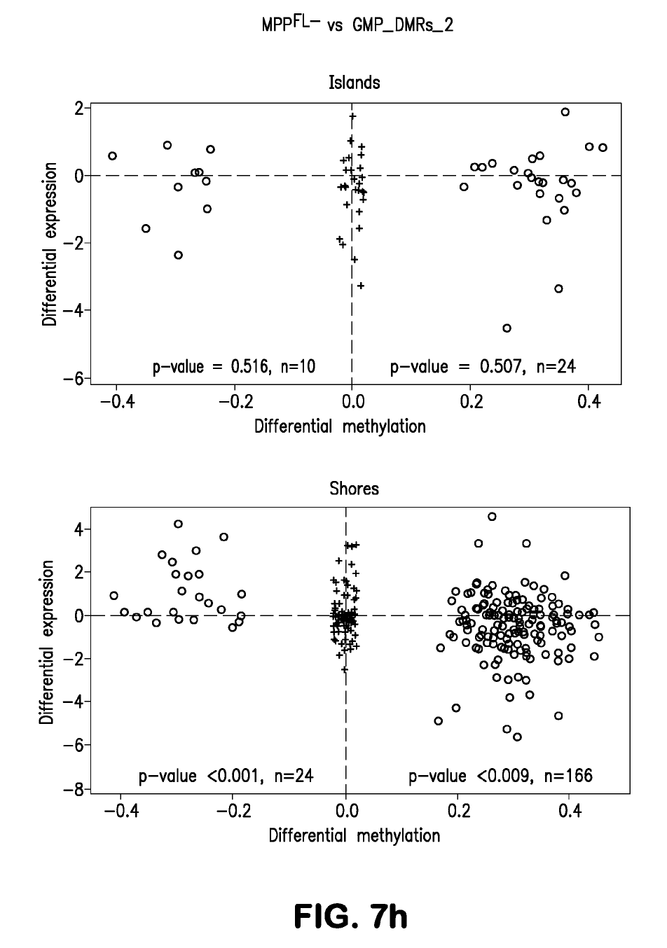

Consistent with previous studies, most DMRs were in CpG island shores (Table 1). The exceptions were for MPP^{FL-} vs. MPP^{FL+}, and MPP^{FL+} vs. CLP, in which most DMRs were in CpG islands: interestingly, both of these transitions are involved in early differentiation. Differential DNA methylation and gene expression showed a statistically significant inverse relationship particularly at CpG island shores (FIG. 2 and FIG. 7).

Gene expression correlates strongly with DMRs at shores as shown in FIGS. 2 and 7. DMRs within 2 kb of gene TSSs (black circles) were divided into two groups: 1) Island (inside, cover, or overlap more than 50% of a CpG island); and 2) Shores (up to 2000 bp away from a CpG island). After RMA preprocessing, the log₂ ratios of the gene expression differences (from left to right) were plotted against Δp (left group minus right group). Black pluses represent random DMR-gene pairs more than 2 kb apart. Wilcoxon rank-sum tests were performed to test the null hypothesis. FIG. 2a depicts MPP^{FL-} vs. DN3_DMRs, while FIG. 2b depicts MPP^{FL-} vs. GMP_DMRs. FIG. 7a depicts MPP^{FL-} vs. MPP^{FL+}_DMRs. FIG. 7b depicts MPP^{FL+} vs. CMP_DMRs. FIG. 7c depicts MPP^{FL+} vs. CLP_DMRs. FIG. 7d depicts CLP vs. DN1_DMRs. FIG. 7e depicts DN1 vs. DN2 DMRs. FIG. 7f depicts DN2 vs. DN3_DMRs. FIG. 7g depicts MPP⁻ vs. DN3_DMRs_{—}2 (CHARM 1.1). FIG. 7h depicts MPP^{FL-} vs. GMP_DMRs_{—}2 (CHARM 1.1).

As the CHARM array design was targeted toward CpG density but not gene architecture per se, a new array was created that included all promoters, and hybridized DNA from three of the groups studied earlier. Analysis showed a similar statistically significant inverse relationship between differential DNA methylation and gene expression, again particularly at CpG island shores (FIG. 7g-h). Thus, CpG island shores are the regions with the most variability in DNA methylation between hematopoietic populations, and this variability correlates best with changes in gene expression. [However, not all DNA methylation changes correlated with changes in gene expression: for example, Tha1 is demethylated during lymphoid specification (see CHARM plots at charm.jhmi.edu/hsc), but is expressed at high levels from MPP through DN3. In converse, and as expected since there are multiple mechanisms for epigenetic regulation, lineage-specifying genes with changes in expression levels were identified, but not in DNA methylation, such as Gata3 and Hes1 (see microarrays deposited in GEO).]

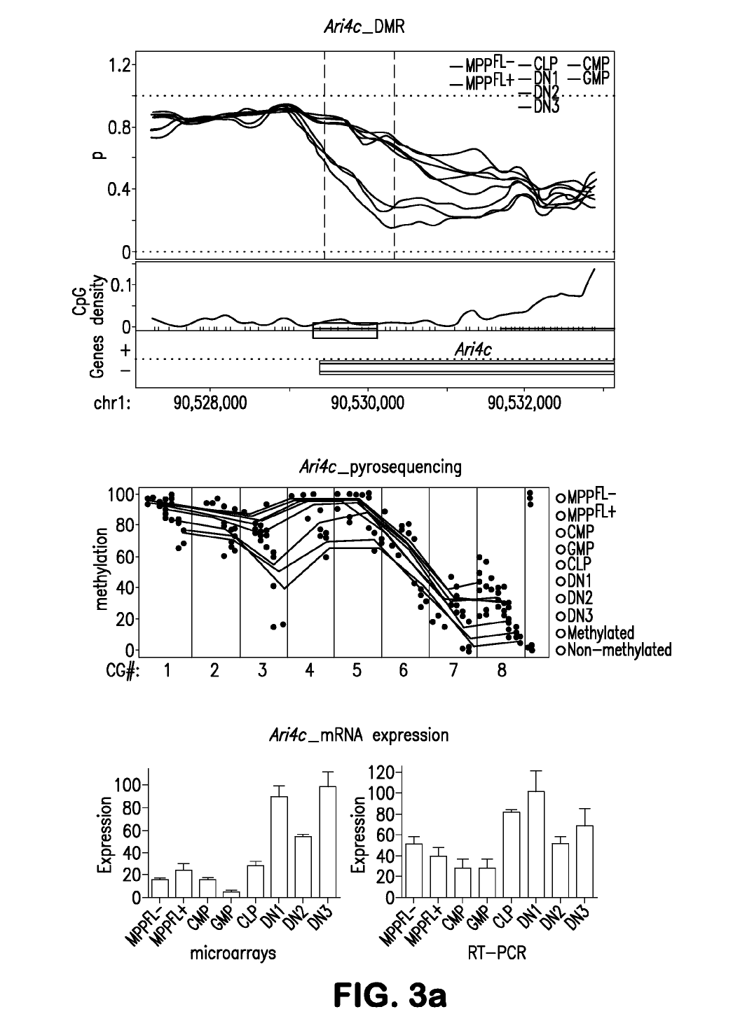

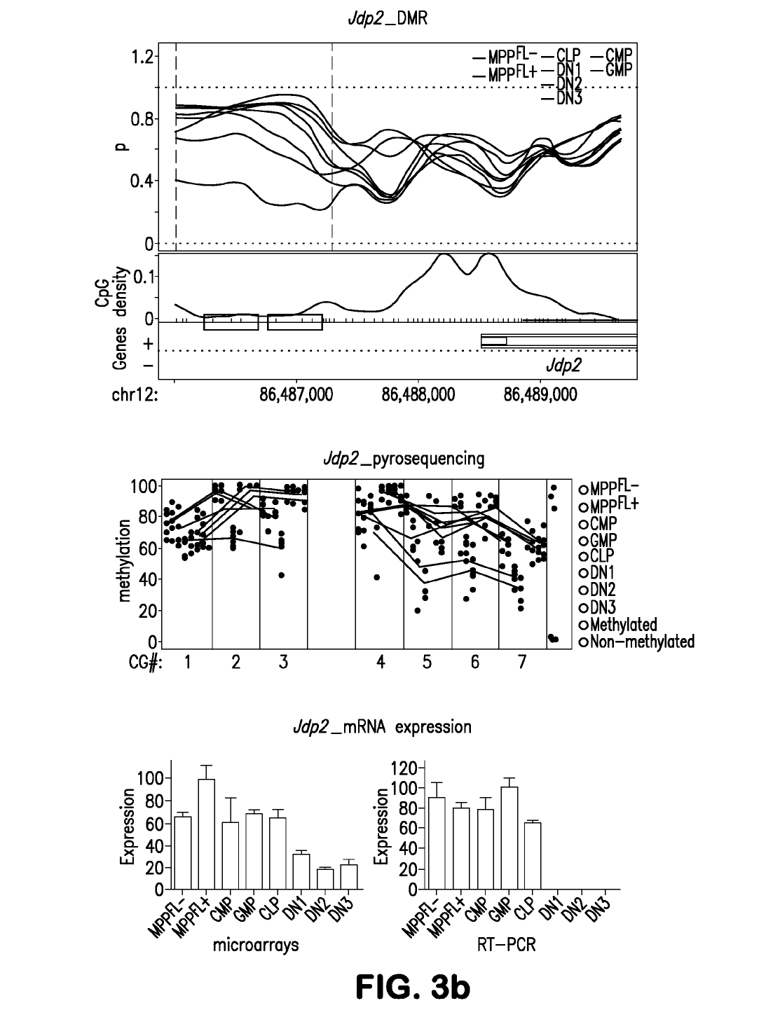

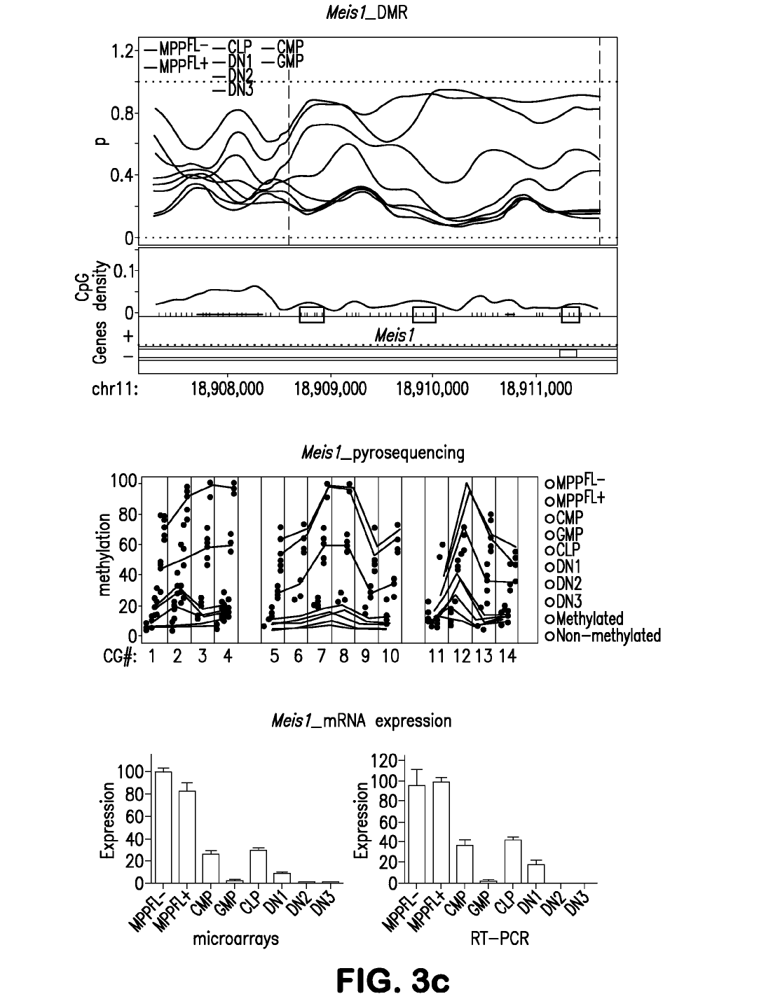

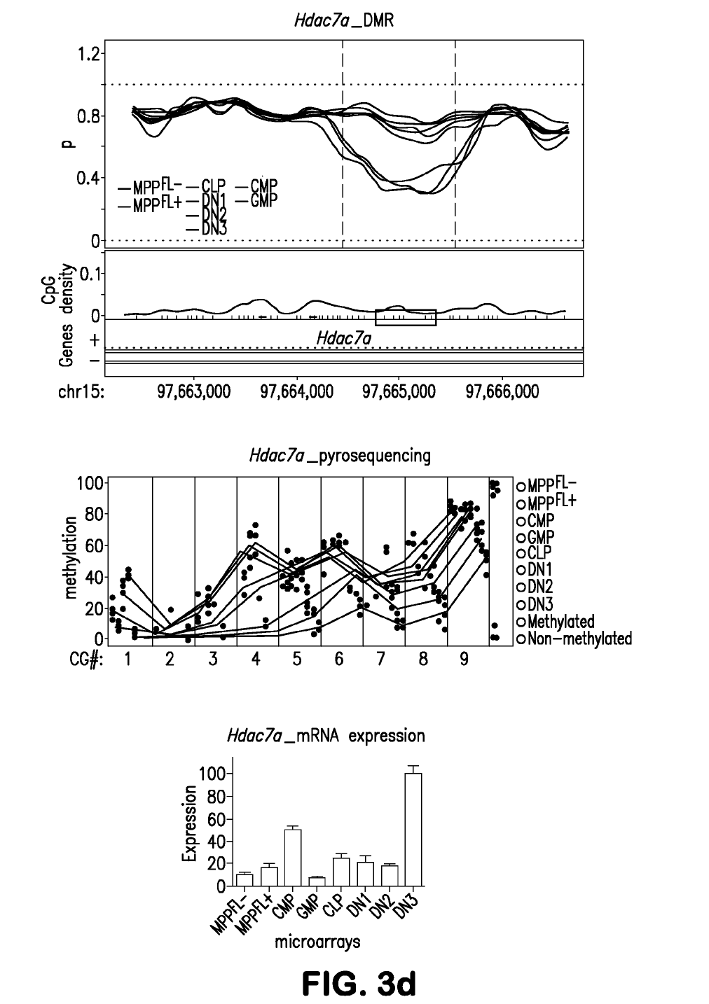

Many novel genes with the potential to contribute to myeloid/lymphoid fate specification were revealed by comparing CHARM-identified DMRs with gene expression data. For example, Arl4c, a member of the ADP-ribosylation factor family of GTP-binding proteins, was upregulated and hypomethylated in DN1-3 thymocytes (FIG. 3a). FIG. 3 shows CHARM identified genes with previously unknown functions in lymphoid/myeloid lineage commitment and pluripotency maintenance.

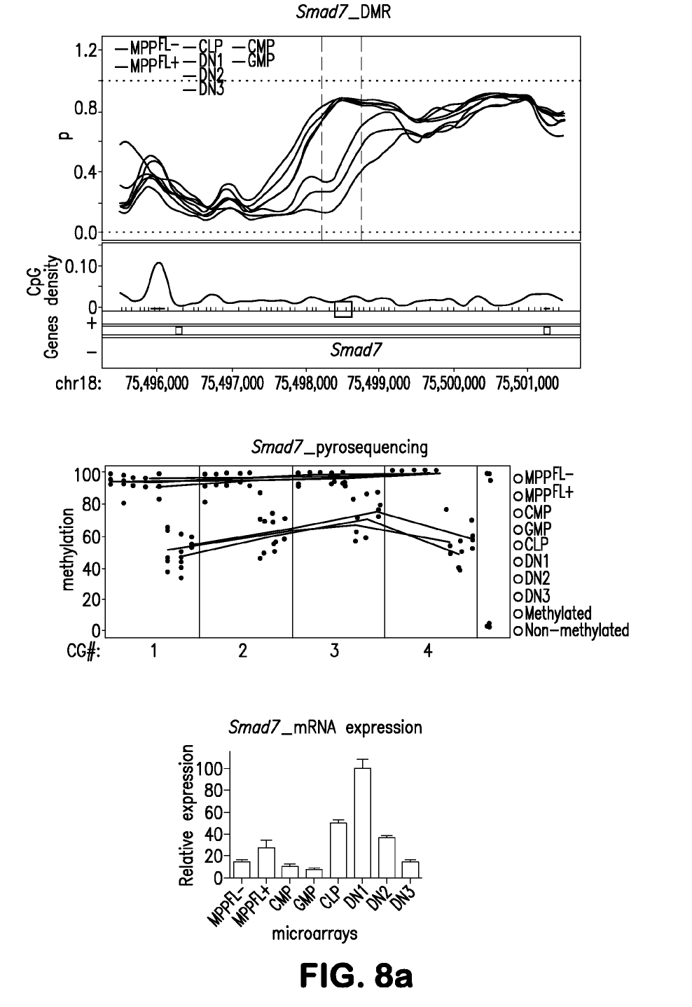

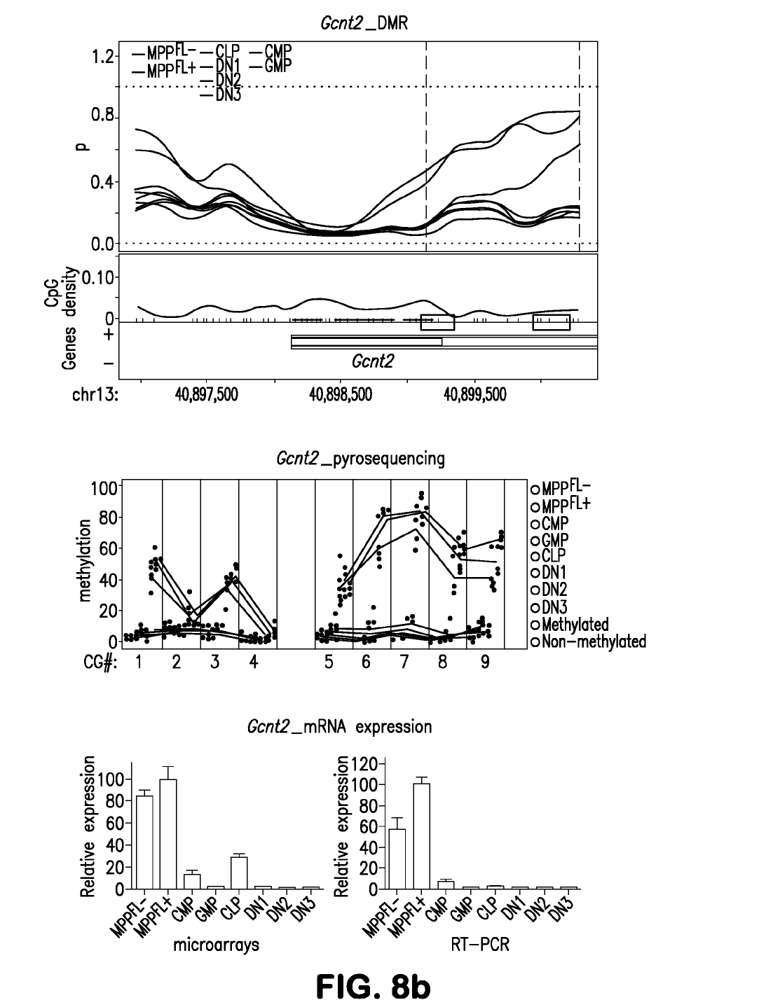

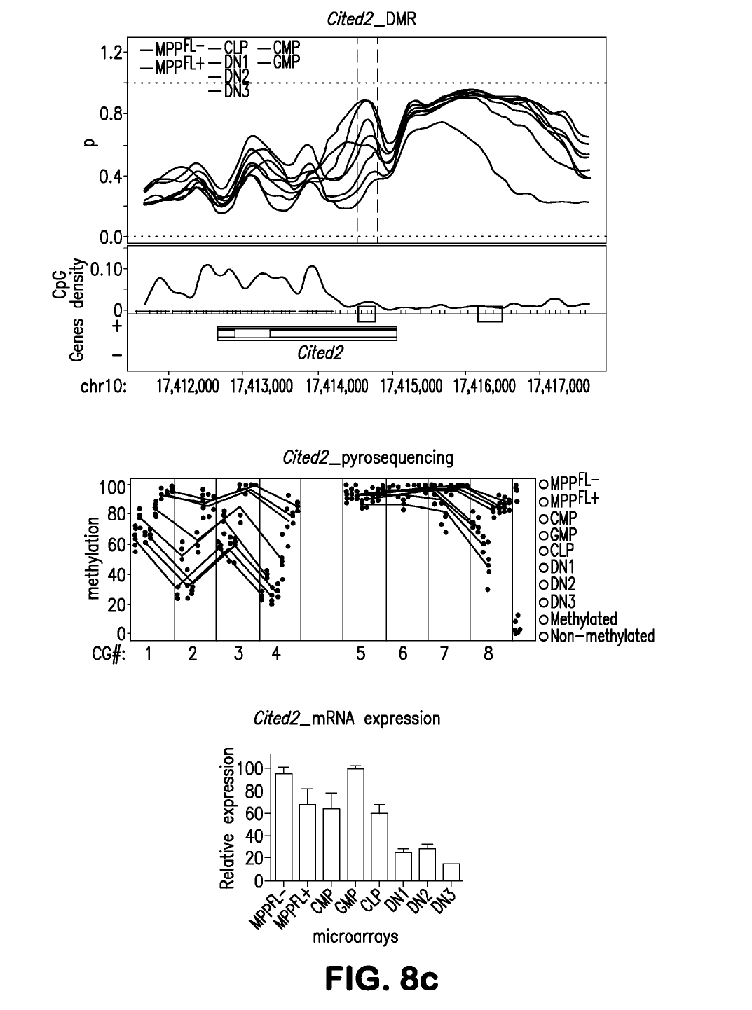

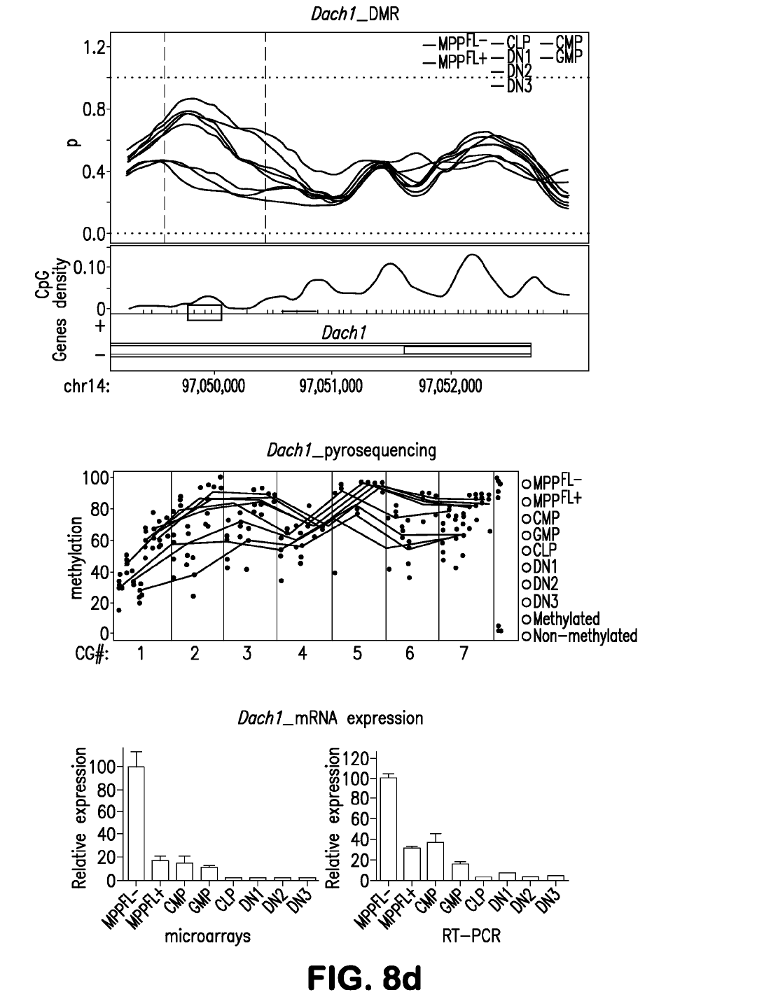

Arl4c may play a role in vesicular transport, but its role in lymphoid specification is unknown. Multiple other genes with DMRs suggestive of a role in lymphoid development, such as Smad7, Gcnt2 and Cited2, were also identified (FIG. 8). Examples of DMRs with DNA methylation changes in lymphocyte/myeoloid progenitors during hematopoiesis are shown in FIG. 8.

Smad7, which negatively regulates TGF-beta signaling, is selectively upregulated and hypomethylated at the earliest stages of thymocyte development, suggesting a role in promoting lymphopoiesis (FIG. 8a). However, it causes myeloid lineage skewing when overexpressed in human cord blood progenitors. Gcnt2 transcripts were downregulated in thymocyte progenitors, and the locus became hypermethylated progressively in DN1-3 progenitors (FIG. 8b), consistent with a role for Gcnt2 in enabling the myeloid potential that is lost during final lymphoid lineage commitment at the DN3 stage.

Novel potential regulators of myelopoiesis were also identified. The Jdp2 locus was hypomethylated and its transcript was upregulated in CMP and GMP relative to thymocyte progenitors (FIG. 3b). Jdp2 is thought to repress transcription by recruiting histone deacetylases and regulating nucleosome assembly. Dach1 was also hypomethylated and expressed from MPP^{FL-} through GMP, but was silenced in CLP and DN1-3 thymocyte progenitors (FIG. 8d), suggesting it may contribute to myelopoiesis. Dach1 has been implicated in transcriptional repression through association with histone deacetylases and its drosophila homolog is known to play a role in gonadal, limb, and ocular development. Thus, Jdp2 and Dach1 may feedback on the epigenome to control expression of tissue specific genes, but their role in hematopoiesis remains uncharacterized.

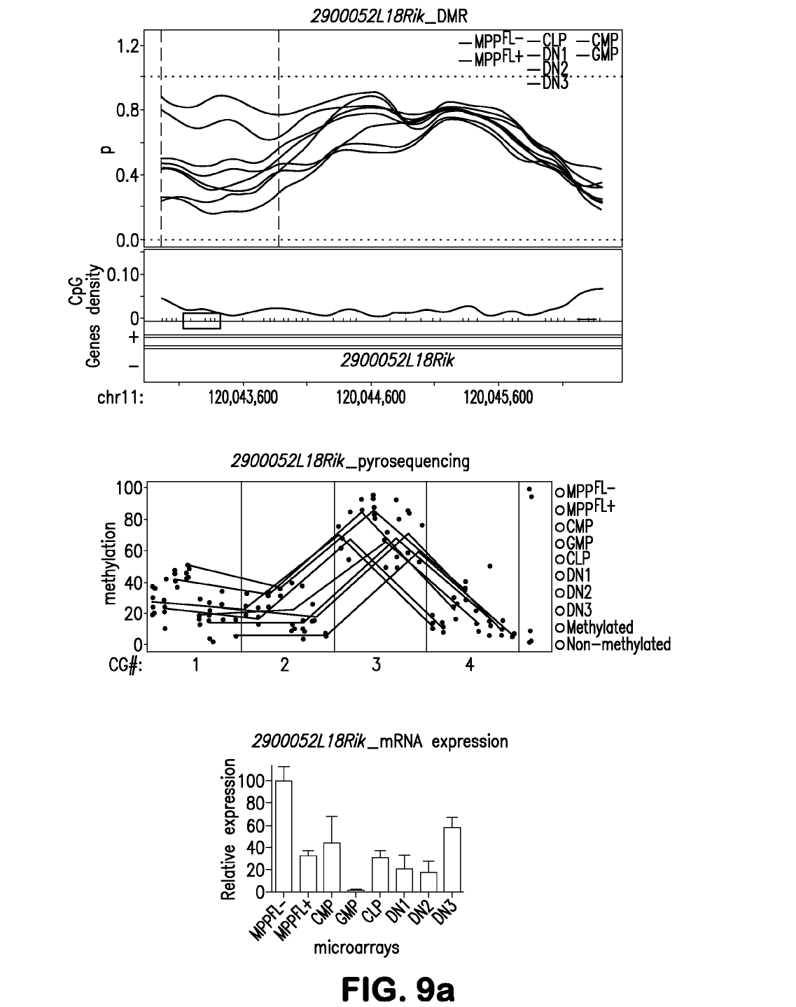

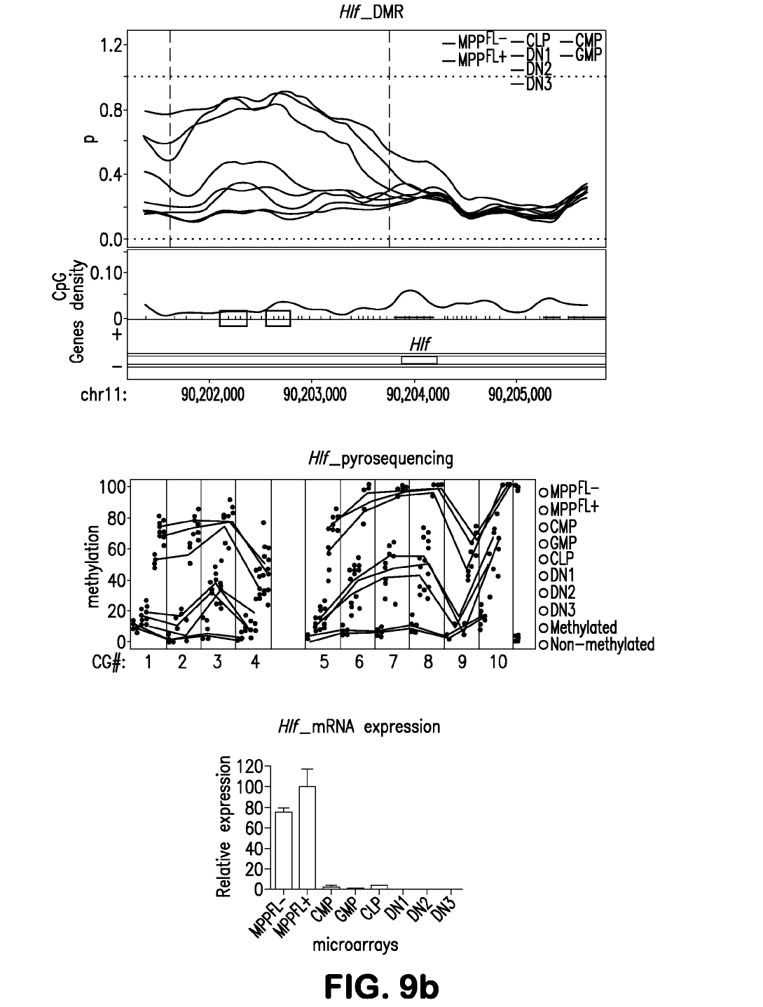

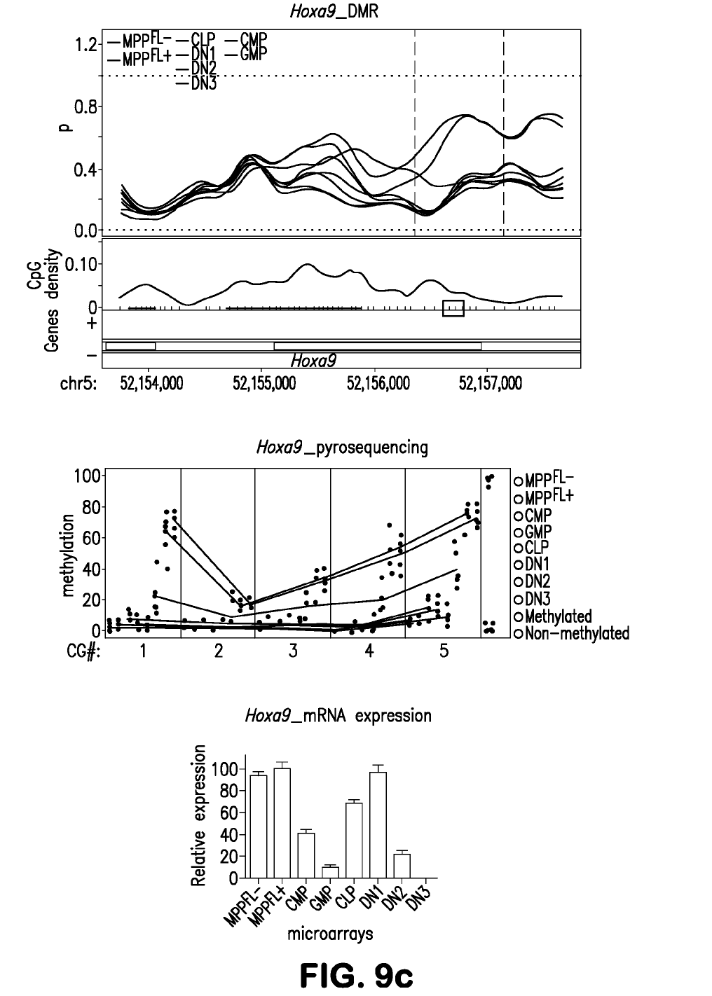

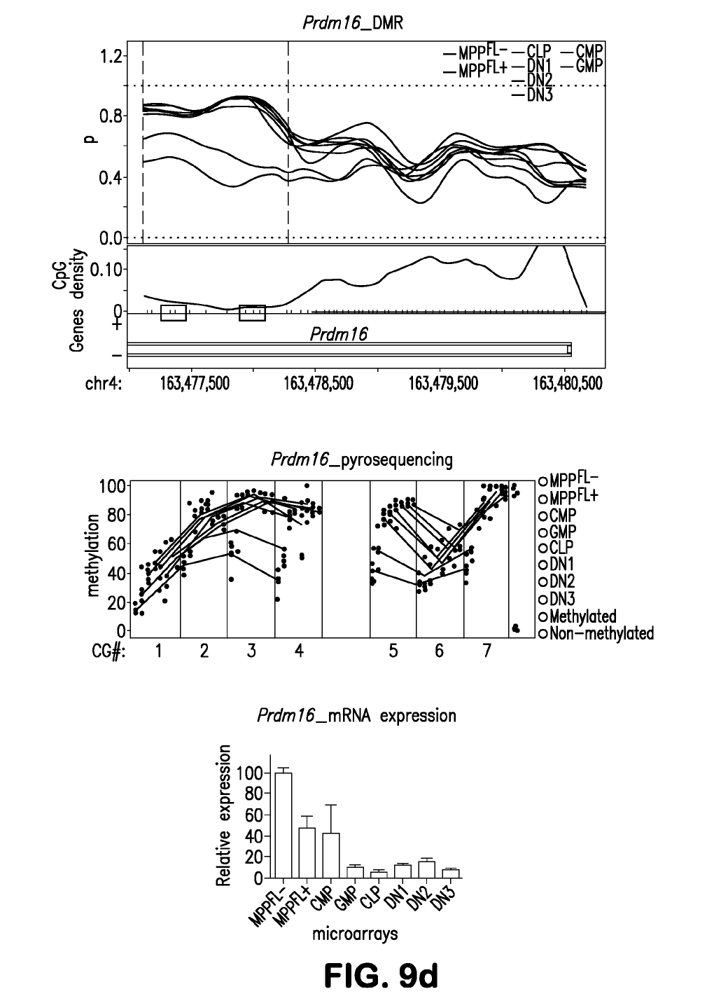

The analyses also revealed a set of genes that were progressively hypermethylated and transcriptionally silenced as differentiation progressed towards both myeloid and lymphoid fates, suggesting a role in maintenance of a multipotent state. Meis1, 2900052L18Rik, Hlf, Hoxa9 and Prdm16 are all such candidates (FIG. 3c and FIG. 9). Meis1 is known to be required for hematopoiesis and megakaryocyte lineage development and may function cooperatively with Hoxa9 to regulate hematopoiesis. Furthermore, both Hlf and Prdm16 have been implicated in hematopoiesis.

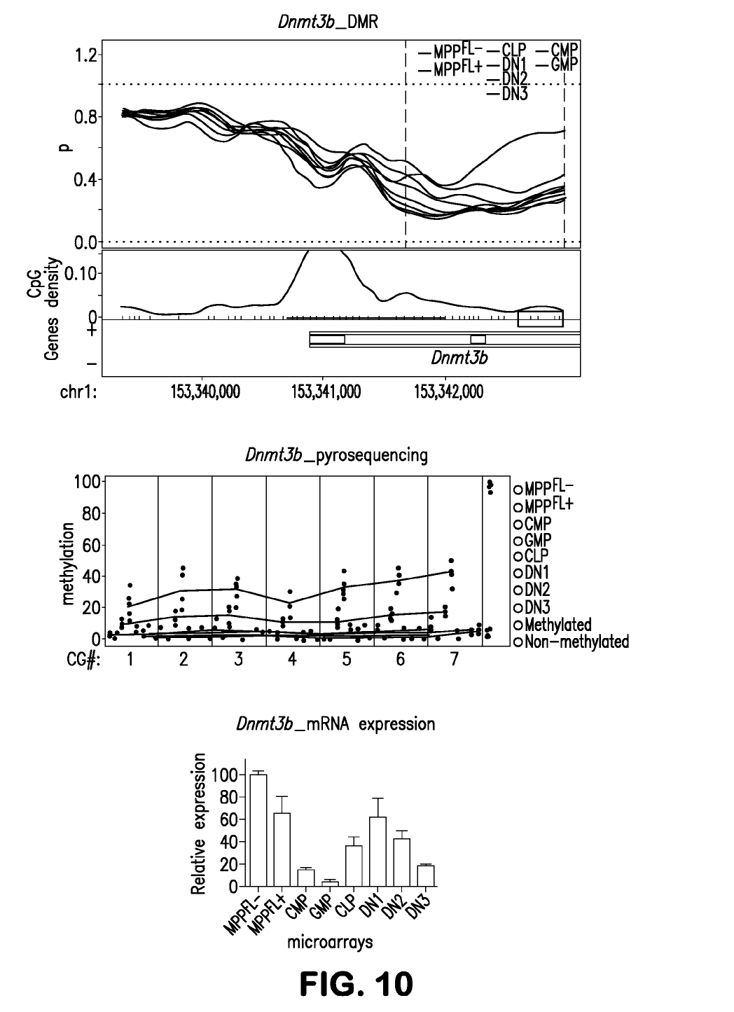

Lastly, epigenetic chromatin modifiers, including Hdac7a and Dnmt3b, were also differentially methylated during hematopoietic differentiation, suggesting feed-forward mechanisms that could expand and lock in epigenetic programming necessary for cell fate commitment (FIG. 3d and FIG. 10). Hdac7, which encodes a histone deacetylase and represses transcription, was demethylated and upregulated in DN1-DN3 thymocytes (FIG. 3d). Since Hdac7 is highly expressed in DN3 cells, which can no longer be reprogrammed toward a myeloid fate by ectopic IL-2R signaling, it may actively repress genes responsible for maintaining myeloid lineage potential. In contrast, Dnmt3b, a methyltransferase responsible for de novo CpG methylation, is hypermethylated and downregulated progressively in CMPs and GMPs (FIG. 10). Dnmt3a and Dnmt3b were shown to be essential for HSC self-renewal, but their roles in lineage commitment remain inconclusive. Downregulation of Dnmt3b in myeloid committed cells could prevent new DNA methylation, helping to maintain the observed hypomethylated state associated with myelopoiesis. In addition, the upregulation of Dnmt3b in DN1 independent of DNA methylation changes might explain the dramatic acquisition of DNA methylation from CLP to DN1 (Table 1).

In summary, these data provide a comprehensive map of the methylome during myeloid and lymphoid commitment from hematopoietic progenitors. To facilitate the general accessibility of the methylome for these hematopoietic progenitors, a novel web platform with which the methylation status of any genomic locus of interest can be easily queried to generate output methylation plots is provided. In addition to identifying candidate genes for further investigation, the data suggest several important themes for the epigenetics of lineage-specific differentiation. First, myelopoiesis and lymphopoiesis achieve markedly different methylation endpoints in differentiation, with lymphopoiesis depending much more heavily on the acquisition of DNA methylation marks, and myelopoiesis depending much more on their loss. Besides providing a mechanism for the proposed DNMT1-dependence of lymphopoiesis, these results may also explain the therapeutic specificity of DNA demethylating drug treatment of myelodysplasia, in which malignant cells arrested in early development may be induced to differentiate by DNA demethylation. In addition, the results show a remarkable dynamic plasticity in methylation during lineage development. The changes are evocative of Waddington's illustrations of hills and valleys in the epigenetic landscape of development. It was recently proposed that development depends on dynamic stochastic variation in the epigenetic landscape in a given genetic environment, and the maturation of undifferentiated progenitors to progressively more differentiated states could restrict that variation. memory in these iPS cells consistent with the DNA methylation profiles described in this paper.

Tables. The following Tables are referred to herein.

Lengthy table referenced here
US20130281304A1-20131024-T00001
Please refer to the end of the specification for access instructions.

Although the invention has been described with reference to the above example, it will be understood that modifications and variations are encompassed within the spirit and scope of the invention. Accordingly, the invention is limited only by the following claims.

LENGTHY TABLES
The patent application contains a lengthy table section. A copy of the table is available in electronic form from the USPTO web site (http://seqdata.uspto.gov/?pageRequest=docDetail&DocID=US20130281304A1). An electronic copy of the table will also be available from the USPTO upon request and payment of the fee set forth in 37 CFR 1.19(b)(3).

1. A method of identifying the differentiation potential of a cell comprising:
comparing the methylation status of one or more nucleic acid sequences of a cell to a known methylation status of the one or more nucleic acid sequences of a reference progenitor cell, wherein a similarity or a difference in methylation status between the cell and the reference cell is indicative of the differentiation potential of the cell.

2. The method of claim 1, with the proviso that the one or more nucleic acid sequences are outside of a promoter region of a gene and outside of a CpG island, and wherein the nucleic acid sequences are up to about 2 kb in distance from a CpG island.

3. The method of claim 2, wherein the one or more nucleic acid sequences are within a gene.

4. The method of claim 2, wherein the one or more nucleic acid sequences are upstream or downstream of a gene.

5. The method of claim 1, wherein the one or more nucleic acid sequences are selected from the group consisting of differentially methylated region (DMR) sequences as set forth in Tables 2a to 2h, FIGS. 1, 3, 5, 8, 9, 10 and any combination thereof.

6. The method of claim 1, wherein the methylation status is performed by one or more techniques selected from the group consisting of a nucleic acid amplification, polymerase chain reaction (PCR), methylation specific PCR, bisulfite pyrosequencing, single-strand conformation polymorphism (SSCP) analysis, restriction analysis, microarray technology, and proteomics.

7. The method of claim 1, wherein the reference progenitor cell is a multipotent progenitor (MPP) cell or an oligopotent progenitor.

8. The method of claim 7, wherein the reference progenitor cell is an oligopotent progenitor selected from a common lymphoid progenitor (CLP) or a common myeloid progenitor (CMP).

9. The method of claim 1, wherein the reference progenitor cell is a granulocyte/macrophage progenitor (GMP), a thymocyte progenitor, or a megakaryocyte-erythrocyte progenitor (MEP).

10. A method of modifying the lineage restriction of a partially or terminally differentiated myeloid or lymphoid cell comprising contacting a partially or terminally differentiated myeloid or lymphoid cell with an agent which alters regulation of the expression or expression product of a gene known to be associated with the differentiation potential of the cell, thereby modifying the lineage restriction of the cell.

11. The method of claim 10, wherein the agent alters regulation of the expression or expression product of a gene set forth in Tables 2a to 2h, Tables 3a to 3h, FIGS. 1, 3, 5, 8, 9, 10 and any combination thereof.

12. The method of claim 10, wherein the agent is a demethylating agent.

13. The method of claim 12, wherein the demethylating agent is a DNA (cytosine-5)-methyltransferase 1 (DNMT1) inhibitor.

14. The method of claim 12, wherein the demethylating agent is a cytidine analog.

15. The method of claim 14, wherein the demethylating agent is agent is 5-azacytidine, 5-aza-2-deoxycytidine.

16. The method of claim 12, wherein the demethylating agent is agent is zebularine.

17. The method of claim 12, wherein the agent is a vector comprising a nucleic acid sequence encoding a gene or portion thereof.

18. The method of claim 12, wherein the agent is a polynucleotide, polypeptide, or small molecule.

19-21. (canceled)

22. A method of inducing myeloid differentiation of a progenitor cell, comprising contacting a progenitor cell with a demethylating agent, thereby inducing myeloid differentiation of the progenitor cell.

23. The method of claim 22, wherein the demethylating agent is a DNA (cytosine-5)-methytransferase 1 (DNMT1) inhibitor.

24. The method of claim 22, wherein the demethylating agent is a cytidine analog.

25. The method of claim 24, wherein the demethylating agent is agent is 5-azacytidine, 5-aza-2-deoxycytidine.

26. The method of claim 22, wherein the demethylating agent is agent is zebularine.

27. The method of claim 22, wherein the progenitor cell is a multipotent progenitor (MPP), a common lymphoid progenitor (CLP), or athymic T cell progenitor.

28. (canceled)

29. A method of differentiating a progenitor cell comprising contacting a progenitor cell with an agent that alters regulation of the expression or expression product of a gene known to be associated with the differentiation potential of the cell, thereby differentiating the progenitor cell.

30. The method of claim 29, wherein the agent alters regulation of the expression or expression product of a gene set forth in Tables 2a to 2h, Tables 3a to 3h, FIGS. 1, 3, 5, 8, 9, 10 and any combination thereof.

31. The method of claim 29, wherein the agent is a demethylating agent.

32. The method of claim 31, wherein the demethylating agent is a DNA (cytosine-5)-methyltransferase 1 (DNMT1) inhibitor.

33. The method of claim 31, wherein the demethylating agent is a cytidine analog.

34. The method of claim 33, wherein the demethylating agent is agent is 5-azacytidine 5-aza-2-deoxycytidine.

35. The method of claim 31, wherein the demethylating agent is agent is zebularine.

36. The method of claim 29, wherein the agent is a vector comprising a nucleic acid sequence encoding a gene or portion thereof.

37. The method of claim 29, wherein the agent is a polynucleotide, polypeptide, or small molecule.

38-40. (canceled)

41. The method of claim 29, wherein expression of at least one of Meis1, Prdm16, 2900052L18Rik, Hlf, Hoxa9, or Hoxa6, is decreases as compared to expression before contacting the cell with the agent.

42-45. (canceled)

46. A method of characterizing the methylation status of the nucleic acid of a cell, comprising:
hybridizing labeled and digested nucleic acid of a cell to a DNA microarray comprising at least 2000 nucleic acid sequences;
determining a pattern of methylation from the hybridizing of (a), thereby characterizing the methylation status for the cell,
wherein the one or more nucleic acid sequences are selected from the Tables 2a to 2h, Tables 3a to 3h, FIGS. 1, 3, 5, 8, 9, 10 and any combination thereof.

47. The method of claim 46, further comprising comparing the methylation status profile to a methylation profile from hybridization of the microarray with labeled and digested nucleic acid from a progenitor cell.

48. The method of claim 47, wherein the progenitor cell is a multipotent progenitor (MPP), a common lymphoid progenitor (CLP), or a thymic T cell progenitor.

49. (canceled)

50. The method of claim 46, further comprising performing one or more techniques selected from the group consisting of a nucleic acid amplification, polymerase chain reaction (PCR), methylation specific PCR, bisulfite pyrosequencing, single-strand conformation polymorphism (SSCP) analysis, and restriction analysis.

51. A plurality of nucleic acid sequences, selected from the group consisting of the differentially methylated region (DMR) sequences as set forth in Tables 2a to 2h, FIGS. 1, 3, 5, 8, 9, 10, and any combination thereof.

52. The plurality of nucleic acid sequences of claim 51, wherein the plurality is a microarray.

53-54. (canceled)

55. A method of generating a cell bank comprising:
(a) identifying the differentiation potential of a plurality of cells using the method of claim 1; and
(b) sorting the cells of (a) by differentiation potential.

56. The method of claim 55, wherein differentiation potential is cell lineage specific.

57. A cell bank produced by the method of claim 55.
